(19) United States
(12) Patent Application Publication
(10) Pub. No.: US 2026/0224538 A1
(43) Pub. Date: Aug. 6, 2026

(54) COMPOUNDS, COMPOSITIONS, AND METHODS OF USE TO INHIBIT PROTEIN AGGREGATION

(71) Applicant: Purdue Research Foundation, West Lafayette, IN (US)

(72) Inventor: Jessica Sonia FORTIN, West Lafayette, IN (US)

(21) Appl. No.: 19/153,309

(22) PCT Filed: Feb. 2, 2024
(86) PCT No.: PCT/US2024/014267

§ 371 Date: Aug. 1, 2025

Related U.S. Application Data
(60) Provisional application No. 63/443,272, filed on Feb. 3, 2023.
(60) Provisional application No. 63/461,370, filed on Apr. 24, 2023.

(51) Int. Cl. A61K 31/428; A61K 31/53; C07D 277/62; C07D 417/10; C07D 417/12

(52) U.S. Cl. CPC A61K 31/428; A61K 31/53; C07D 277/62; C07D 417/10; C07D 417/12

(57) ABSTRACT

The disclosure relates to, among other things, benzisothiazoles, benzisoxazoles, and indazoles; a pharmaceutical composition comprising such compounds; and a method of inhibiting protein aggregation in a subject having, or at risk for, aggregation of a protein prone to aggregate in a state of disease, which method comprises administering to the subject the composition in an amount effective to inhibit protein aggregation.

### CROSS-REFERENCE TO RELATED APPLICATIONS

This application claims the benefit of U.S. Prov. Appl. No. 63/443,272, filed Feb. 3, 2023; and of U.S. Prov. Appl. No. 63/461,370, filed Apr. 24, 2024, each of which is incorporated by reference as if fully set forth herein.

### GOVERNMENT SUPPORT CLAUSE

This invention was made with government support under contracts AG070447 and AG071985 awarded by the National Institutes of Health. The government has certain rights in the invention.

### TECHNICAL FIELD

The present disclosure relates to, among other things, benzisothiazole, benzisoxazoles, and indazoles, compositions comprising same, and methods of use to inhibit protein aggregation, such as by inhibition of oligomer formation.

### BACKGROUND

Proteins are large macromolecules, which are comprised of long chains of amino acids and play various functional roles throughout the body. Protein structure is critical to function. When the native structure of a protein is altered, the protein can become useless or even detrimental to a cell. Resulting diseases are known as protein folding disorders.

There are at least 41 different nonhomologous proteins that have been identified with high propensity to change conformation and form fibrils that accumulate into extracellular amyloid-like deposits. Fibril formation and build up into extracellular amyloid deposits have been associated with a long list of serious chronic diseases such as AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, and transthyretin amyloidosis. In each disease a different endogenous protein self-assembles into highly ordered fibrillar structures. Although there is no specific sequence homology between these proteins, they all undergo major conformational changes to produce β-sheet structures that strongly tend to aggregate into water-insoluble fibrous polymers.

Much effort has been directed toward understanding and preventing amyloid formation. The molecular mechanism of amyloid formation is still not fully understood, and no general strategy has emerged for developing small molecules that can inhibit the formation of fibrils or disaggregate amyloid deposits in any mammal.

Misfolded proteins can result from sporadic, hereditary, and transmissible causes and can lead to a diverse array of conditions. Disfigured proteins can accumulate in any organ, including the liver, spleen, kidney, and brain, partially attributing to the vast pathological differences in protein misfolding diseases. Despite these differences, the generic mechanism of disease remains the same—as more proteins misfold, they accumulate in clusters known as amyloid plaques. Individual misfolded protein monomers conjoin to form oligomers, which elongate to form amyloid fibrils, which then accumulate extracellularly into deposits during the final state of this process, known formally as amyloidosis. Short fibrils and intermediate species, such as oligomers, have been shown to be cytotoxic. Therefore, it is crucial to find therapeutic strategies to mitigate the formation of oligomers.

Currently, there are no effective therapies for resolving or halting the progression of neurodegenerative diseases associated with protein disorders. One of the best-known protein-misfolding diseases is the neurodegenerative condition known as Alzheimer's disease (AD). AD is associated with the formation and accumulation of amyloid-β (Aβ) in the brain as well as tangle formation due to misfolding of the tubulin associate unit (tau) protein. Another important misfolding protein, α-synuclein (α-syn), is highly involved in the pathophysiology of Parkinson's Disease (PD). When α-syn misfolds, it aggregates and forms inclusions within the neurons called Lewy bodies. Lewy bodies lead to cell lysis, which may spread to other neurons via the synaptic cleft, massive inflammation, and subsequent disease.

One widely accepted therapeutic approach to dealing with misfolded proteins is the use of small molecules as stabilizers. In the past small molecule drugs, such as tafamidis, have been used to stabilize the transthyretin (TTR) transport protein. TTR is a protein that is mostly formed in the liver and plays a key role in the progression of amyloid fibrils after the dissociation of the TTR tetramer into monomers, which then unfold into oligomers. These oligomers act as seeds for fibril growth, paving the way for the classic presentation of amyloidosis.

There remains, however, a long-felt and currently unmet need for small molecule drugs that can inhibit α-syn and tau, specifically tau isoform 2N4R (tau 2N4R), aggregation, which also contribute to neurodegeneration. In view of the foregoing, it is an object of the present disclosure to provide such small molecule drugs. This and other objects and advantages, as well as inventive features, will be apparent from the detailed description provided herein.

### SUMMARY

Provided is a compound comprising a benzisothiazole linked to an amide, a formamide, an amine, or a urea, or a pharmaceutically acceptable salt thereof, wherein at least one carbon on the benzyl ring of the benzisothiazole is substituted with a substituent selected from the group consisting of a C₁-C₆ alkyl, —NO₂, and a halo. The C₁-C₆ alkyl can be methyl. The halo can be F, Cl, or Br. The compound can have the structure:

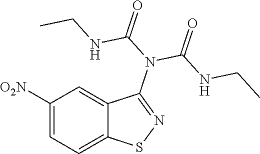 [SMILES: CCNC(=O)N(C(=O)NCC)c1nsc2ccc([N+](=O)[O-])cc12]

or a pharmaceutically acceptable salt thereof.

The compound can have the structure:

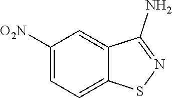 [SMILES: Nc1nsc2ccc([N+](=O)[O-])cc12]

or a pharmaceutically acceptable salt thereof.

The compound can have the structure:

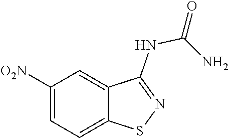 [SMILES: NC(=O)Nc1nsc2ccc([N+](=O)[O-])cc12]

or a pharmaceutically acceptable salt thereof.

The compound can have the structure:

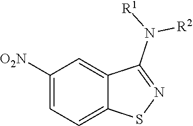

or a pharmaceutically acceptable salt thereof;
wherein each R¹ and R² is independently selected from hydrogen, alkyl, cycloalkyl, aryl, heteroaryl, —SO₂R, and —C(O)R, wherein R₁ and R₂ are each, independently, optionally substituted with an alkyl, a nitro, a cyano, a halo, an amino, a hydroxy, an alkoxy, an aryl, or a heteroaryl, and wherein R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R is optionally substituted with a halo, an amino, a nitro, a cyano, a hydroxy, an alkoxy, an aryl, or a heteroaryl group.

An embodiment of the above formula is a compound having the structure:

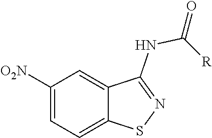

or a pharmaceutically acceptable salt thereof;
wherein R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R can be optionally substituted with a halo, an amino, a nitro, a cyano, a hydroxy, an alkoxy, an aryl, or a heteroaryl group. In an embodiment, R is —CH₂—Cl.

Further provided is a compound having the structure:

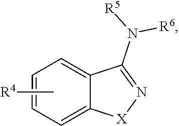

such as

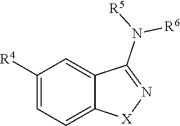

or a pharmaceutically acceptable salt thereof;
wherein:
X is O, NR³, or S, wherein R³ is H or alkyl (e.g., C₁₋₆ alkyl);
R⁴ is an electron withdrawing group, such as halo, —NO₂, —CN, —CF₃, —C(O)R″, —NC(O)R″ or —NSO₂R″ wherein R″ can be H, C₁₋₆ alkyl and aryl; and
each R⁵ and R⁶ is independently selected from hydrogen, alkyl, cycloalkyl, aryl, heteroaryl, —SO₂R, —C(O)NHR, and —C(NR⁷)NHR, wherein R⁵ and R⁶ can each be independently optionally substituted with an alkyl, a nitro, a cyano, a halo, an amino, a hydroxy, an alkoxy, an aryl, or a heteroaryl (e.g. thiophene or pyrrole), and R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R can be optionally substituted with a halo, an amino, a nitro, a cyano, a hydroxy, an alkoxy, an aryl, or a heteroaryl group.

Further provided is a compound having the structure:

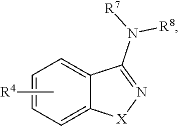

such as

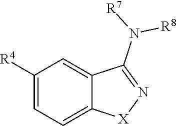

or a pharmaceutically acceptable salt thereof;
wherein:
X is O, NR³, or S, wherein R³ is H or alkyl (e.g., C₁₋₆ alkyl);
R⁴ is an electron withdrawing group, such as halo, —NO₂, —CN, —CF₃, —C(O)R″, —NC(O)R″ or —NSO₂R″ wherein R″ can be H, C₁₋₆ alkyl and aryl; and
each R⁷ and R⁸ is independently selected from H, —C(O)NHR, alkylaminoalkyl, aminoalkyl, arylamino, heteroarylamino, alkyl substituted with at least one group comprising a hydrogen atom capable of hydrogen bonding with at least one hydrogen bonding acceptor, aryl substituted with at least one group comprising a hydrogen atom capable of hydrogen bonding with a hydrogen bonding acceptor, heteroaryl substituted with at least one group comprising a hydrogen atom capable of hydrogen bonding with a hydrogen bonding acceptor, and heteroaryl comprising at least one hydrogen atom capable of hydrogen bonding with a hydrogen bonding acceptor, each of which can independently optionally be substituted with an alkyl, a nitro, a cyano, a halo, an amino, a hydroxy, an alkoxy, an aryl, or a heteroaryl, and R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl and wherein R can be optionally substituted with a halo, an amino, a nitro, a cyano, a hydroxy, an alkoxy, an aryl, or a heteroaryl group.

In one example, R⁷ is H and R⁸ is selected from H, —C(O)NHR, alkylaminoalkyl, aminoalkyl, arylamino, and heteroarylamino, each of which can be optionally substituted with a halo, an amino, a nitro, a cyano, a hydroxy, an alkoxy, an aryl, or a heteroaryl group.

Further provided is a pharmaceutical composition comprising at least one of the above-described compounds, or pharmaceutically acceptable salts thereof, and a pharmaceutically acceptable carrier.

Still further provided is a method of inhibiting protein aggregation in a subject having, or at risk for, aggregation of a protein prone to aggregate in a state of disease, the method comprising administering to the subject one or more of the above-described compounds, or pharmaceutically acceptable salts thereof (e.g., in a pharmaceutical composition comprising one or more of the above-described compounds, or pharmaceutically acceptable salts thereof, and a pharmaceutically acceptable carrier), in an amount effective to inhibit protein aggregation. The protein prone to aggregate can be one or more of islet amyloid polypeptide, amyloid-β, α-synuclein, tubulin associated unit (tau), or transthyretin. The tau can be tau isoform 2N4R or 1N4R or 0N4R, 2N3R or 1N3R or 0N3R with or without post-translational changes, such as phosphorylation. The disease can be AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis. The subject can have, or be at risk for, Alzheimer's disease. The subject can have, or be at risk for, Parkinson's disease.

### DESCRIPTION OF THE DRAWINGS

The drawings illustrate generally, by way of example, but not by way of limitation, various embodiments discussed herein.

FIG. 1 shows representative structures of BTA and its derivatives: urea (1), thiourea (2), sulfonamide (3) and triazole (4). The original compound is designated in blue, and the derivative structural modifications are indicated in red and green.

FIGS. 2A-2J shows data that indicate BTA is a general inhibitor of fibril formation. (A) A bar graph depicting the arbitrary maximum fluorescence intensity in percentage for fibril type including IAAP, Aβ₁₋₄₀, Aβ₁₋₄₂, α-synuclein and TTR₈₁₋₁₂₇. Since IAPP, α-synuclein, and TTR₈₁₋₁₂₇ had arbitrary percent fluorescence under 40% for both resveratrol BTA treatments, analysis via electron micrograph (EM) was performed. The error bars represent the individual standard deviation of the mean for each trial. (B-J) EM of peptides incubated with either 100 μM resveratrol, 100 μM BTA, or 0.1% DMSO control at 37° C. for 1 hour (10 μM of IAPP) in PBS, 24 hours (6 μM of α-synuclein) in PBS, and 24 hours (10 μM of TTR₈₁₋₁₂₇) in sodium acetate buffer, pH 4. (B) IAPP with 0.1% DMSO at 40 k magnification. (C) α-synuclein with 0.1% DMSO at 25 k magnification. (D) TTR₈₁₋₁₂₇ with ≤0.1% DMSO at 40 k magnification. (E) IAPP with 100 μM BTA at 40 k magnification. (F) α-synuclein with 100 μM BTA at 25 k magnification. (G) TTR₈₁₋₁₂₇ with 100 μM BTA at 40 k magnification. (H) IAPP with 100 μM resveratrol at 40 k magnification. (I) α-synuclein with 100 μM resveratrol at 25 k magnification. (J) TTR₈₁₋₁₂₇ with 100 μM resveratrol at 40 k magnification. Scale bars=200 nm.

FIGS. 3A-3D shows data that indicate compounds 1-4 failed to abrogate substantially fibril formation of both TTR fragments (TTR₈₁₋₁₂₇, TTR₁₀₁₋₁₂₅). (A) TTR₈₁₋₁₂₇ fibril formation of BTA derivatives 1-4, resveratrol (positive control) and BTA at 100 μM (molar ratio 1:10) assessed by ThT fluorometric assays in a time-dependent manner in presence of DMSO (0.1%, control; CTRL) (B) TTR₁₀₁₋₁₂₅ fibril formation of BTA derivatives 1-4, resveratrol (positive control), and BTA at 100 μM (molar ratio 1:10) by ThT fluorometric assays in a time-dependent manner. (C) and (D) same as (B) but evaluating compounds at final concentration of 50 μM (molar ratio 1:5) and 25 μM (molar ratio 1:2.5).

FIGS. 4A-4D shows the anti-fibrillary effect of 5-nitro-1,2-benzothiazol-3-amine and several derivatives on different prone-to-aggregate proteins with special emphasis on α-syn and tau isoform 2N4R. (A) Histogram representing Thioflavin T fluorescence intensity of prone-to-aggregate proteins incubated with compounds 5 (negative control) and 5-nitro-1,2-benzothiazol-3-amine (a). (B) Tau isoform 2N4R kinetics of fibril formation in the presence or absence of 5-nitro-1,2-benzothiazol-3-amine (a), compound 10, and compound 13. 5-nitro-1,2-benzothiazol-3-amine and compound 13 delayed the lag time. (C) Dose dependency of 5-nitro-1,2-benzothiazol-3-amine on α-syn. (D) Similar dose dependency response but applied tau isoform 2N4R. For each concentration (3.125, 6.25, 12.5, 25, 50, and 100 μM), triplicate data were collected at the plateau phase for α-syn and at 15 hours for tau. Each protein was tested at 6 μM. The resulting molar ratios (protein: compound) consist of ~1:0 (control DMSO), 1:0.5 (compound at 3.125 μM), 1:1 (compound at 6.25 μM), 1:2 (compound at 12.5 μM), 1:4 (compound at 25 μM), 1:8 (compound at 50 μM), and 1:16 (compound at 100 μM).

FIGS. 5A-5E shows 5-nitro-1,2-benzothiazol-3-amine (a) reduced α-syn and tau isoform 2N4R oligomer formation by photo-induced cross-linking of unmodified proteins (PICUP). (A) α-Syn (6 μM) was cross-linked (PICUP assay) with different compounds at 50 μM (~molar ratio, 1:5). (B) Tau isoform 2N4R (6 μM) was cross-linked (PICUP assay) with different compounds at 50 μM (~molar ratio, 1:5). (C) Dose-dependent inhibitory activity of 5-nitro-1,2-benzothiazol-3-amine (compound a) on α-syn oligomerization. The protein (6 μM) was incubated with 5-nitro-1,2-benzothiazol-3-amine (a): 50 μM (molar ratio 1:8), 25 μM (molar ratio 1:16), 12.5 μM (molar ratio ~1:32), 6.25 μM (molar ratio ~1:64), 3.125 μM (molar ratio ~1:128). (D) Similar conditions were applied to evaluate the dose-dependent inhibitory activity on tau isoform 2N4R. (E) Dose-dependent inhibitory activity of 5-nitro-1,2-benzothiazol-3-amine derivative with highest anti-aggregation activity, i.e., compound 13, on α-syn oligomerization with similar conditions. Compound 13 reduced the oligomer formation at high concentration, i.e., 200 μM. Coomassie blue-stained polyacrylamide gels showed high molecular weight α-syn or tau oligomers with control (0.125% DMSO). 5-nitro-1,2-benzothiazol-3-amine (a) stopped formation of α-syn and tau oligomers (uncross-linked) in a dose-dependent manner. Lower concentration of 5-nitro-1,2-benzothiazol-3-amine (a) showed higher prominence of oligomer formation.

FIG. 6 shows that 5-nitro-1,2-benzothiazol-3-amine (a) reduced α-synuclein (α-syn) and tau fibril formation as validated by transmission electron microscopy (TEM). α-Syn (6 μM) was incubated with DMSO (0.25%; ‘CTRL’) or 5-nitro-1,2-benzothiazol-3-amine (100 μM) for ~80 hours prior to TEM visualization (molar ratio 1:10). Tau isoform 2N4R (10 μM) was incubated with DMSO (0.25%; ‘CTRL’) or 5-nitro-1,2-benzothiazol-3-amine (100 μM) for ~50 hours (i.e., in previously described experiments aimed at monitoring fibril formation by measuring ThT fluorescence) prior to TEM visualization (molar ratio 1:10). High magnifications (40K) showed fewer fibrils in protein samples supplemented with 5-nitro-1,2-benzothiazol-3-amine in comparison with DMSO control. TEM results validated the reduction in fibrils monitored by ThT assays. Scale bars=200 nm.

FIGS. 7A-7C shows that 5-nitro-1,2-benzothiazol-3-amine (a) abrogated the inclusion formation in M17D neuroblastoma cells that express inclusion-prone αS-3K::YFP. (A) Incucyte-based analysis of punctate YFP signals at t=96 hrs, normalized to 0.1% DMSO. 8 independent experiments (N=8) were performed. Student's t-test, ****, p<0.0001, ***, p<0.001, **, p<0.01 (B) Same as panel A, but confluence was plotted. C) M17D cells that express an αS3K::YFP fusion protein (dox-inducible) were treated with 0.1% DMSO (vehicle control) or different concentrations of 5-nitro-1,2-benzothiazol-3-amine (a) at t=24 hrs. Cells were induced with dox at t=48 hrs. Representative images (YFP, left; bright-field, right); scale bar, 25 μm.

FIGS. 8A-8C shows BTA reduced the confluence but not the inclusion formation in M17D neuroblastoma cells that express inclusion-prone αS-3K::YFP. (A) M17D cells that express an αS3K::YFP fusion protein (dox-inducible) were treated with 0.1% DMSO (vehicle control) or 40 μM BTA at t=24 hours. Cells were induced with dox at t=48 hours. Representative images (YFP, left; bright-field, right); scale bar, 25 μm. (B) Incucyte-based analysis of punctate YFP signals at t=96 hours, normalized to 0.1% DMSO. Eight independent experiments (N=8) were performed. Student's t-test, **, p<0.01. (C) Same as panel B, but confluence was plotted (reduction in confluence is indicative of the compound being slightly toxic at this concentration).

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Provided is a compound comprising a benzisothiazole linked to an amide, a formamide, an amine, or a urea, or a pharmaceutically acceptable salt thereof, wherein at least one carbon on the benzyl ring of the benzisothiazole is substituted with a substituent selected from the group consisting of a C₁-C₆ alkyl, —NO₂, and a halo. The C₁-C₆ alkyl can be methyl. The halo can be F, Cl, or Br. The compound can have the structure:

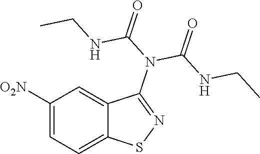 [SMILES: CCNC(=O)N(C(=O)NCC)c1nsc2ccc([N+](=O)[O-])cc12]

or a pharmaceutically acceptable salt thereof.

The compound can have the structure:

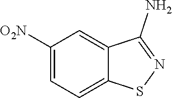 [SMILES: Nc1nsc2ccc([N+](=O)[O-])cc12]

or a pharmaceutically acceptable salt thereof.

The compound can have the structure:

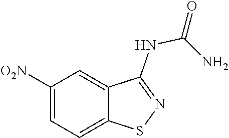 [SMILES: NC(=O)Nc1nsc2ccc([N+](=O)[O-])cc12]

or a pharmaceutically acceptable salt thereof.

The compound can have the structure:

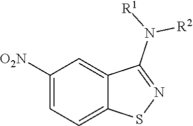

or a pharmaceutically acceptable salt thereof;
wherein each R¹ and R² is independently selected from hydrogen, alkyl, cycloalkyl, aryl, heteroaryl, —SO₂R, and —C(O)R, wherein R¹ and R² can be independently substituted with an alkyl, a nitro, a cyano, a halo, an amino, a hydroxy, an alkoxy, an aryl, or a heteroaryl, and wherein R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R can be substituted with a halo, an amino, a nitro, a cyano, a hydroxy, an alkoxy, an aryl, or a heteroaryl group.

An embodiment of the above formula is a compound having the structure:

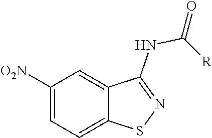

or a pharmaceutically acceptable salt thereof;
wherein R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R can be substituted with a halo, an amino, a nitro, a cyano, a hydroxy, an alkoxy, an aryl, or a heteroaryl group. In an embodiment, R is —CH₂—Cl.

Further provided is a compound having the structure:

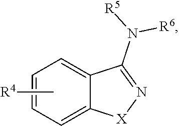

such as

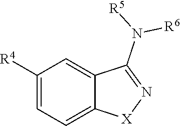

or a pharmaceutically acceptable salt thereof;
wherein:
X is O, NR³, or S, wherein R³ is H or alkyl (e.g., C₁₋₆ alkyl); R⁴ is an electron withdrawing group, such as halo, —NO₂, —CN, —CF₃, —C(O)R″, —NC(O)R″ or —NSO₂R″ wherein R″ can be H, C₁₋₄ alkyl and aryl; and each R⁵ and R⁶ is independently selected from hydrogen, alkyl, cycloalkyl, aryl, heteroaryl, —SO₂R, —C(O)NHR, and —C(NR⁷)NHR, wherein R⁵ and R⁶ can each be independently optionally substituted with an alkyl, a nitro, a cyano, a halo, an amino, a hydroxy, an alkoxy, an aryl, or a heteroaryl, and R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl, and wherein R can be optionally substituted with a halo, an amino, a nitro, a cyano, a hydroxy, an alkoxy, an aryl, or a heteroaryl group.

In one example, R⁵ is H and R⁶ is selected from H, —C(O)NHR, alkylaminoalkyl, aminoalkyl, arylamino, and heteroarylamino. In another example, R⁵ and R⁶ are each independently —C(O)NHR, wherein each R is independently C₁₋₆ alkyl. Thus, for example, R⁵ and R⁶, together with the nitrogen atom to which they are attached, can form the group:

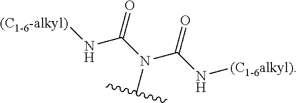

Further provided is a compound having the structure:

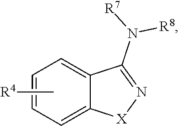

such as

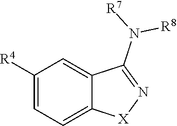

or a pharmaceutically acceptable salt thereof;
wherein:
X is O, NR³, or S, wherein R³ is H or alkyl (e.g., C₁₋₆ alkyl); R⁴ is an electron withdrawing group, such as halo, —NO₂, —CN, —CF₃, —C(O)R″, —NC(O)R″ or —NSO₂R″ wherein R″ can be H, C₁₋₄ alkyl and aryl; and
each R⁷ and R⁸ is independently selected from H, —C(O)NHR, alkylaminoalkyl, aminoalkyl, arylamino, heteroarylamino, alkyl substituted with at least one group comprising a hydrogen atom capable of hydrogen bonding with at least one hydrogen bonding acceptor, aryl substituted with at least one group comprising a hydrogen atom capable of hydrogen bonding with a hydrogen bonding acceptor, heteroaryl substituted with at least one group comprising a hydrogen atom capable of hydrogen bonding with a hydrogen bonding acceptor, and heteroaryl comprising at least one hydrogen atom capable of hydrogen bonding with a hydrogen bonding acceptor, each of which can independently optionally be substituted with an alkyl, a nitro, a cyano, a halo, an amino, a hydroxy, an alkoxy, an aryl, or a heteroaryl, and R is an alkyl (e.g., C₁₋₆ alkyl), an alkenyl (e.g., C₁₋₆ alkenyl), an alkynyl (e.g., C₁₋₆ alkynyl), a cycloalkyl, a heterocycloalkyl, an aryl, or a heteroaryl and wherein R can be optionally substituted with a halo, an amino, a nitro, a cyano, a hydroxy, an alkoxy, an aryl, or a heteroaryl group.

Examples of groups comprising a hydrogen atom capable of hydrogen bonding with a hydrogen bonding acceptor include —C(O)OH, —C(O)NHR, —OH, and the like.

Examples of heteroaryl comprising at least one hydrogen atom capable of hydrogen bonding with a hydrogen bonding acceptor include:

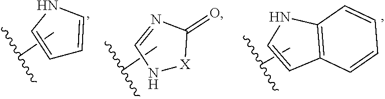

and the like.

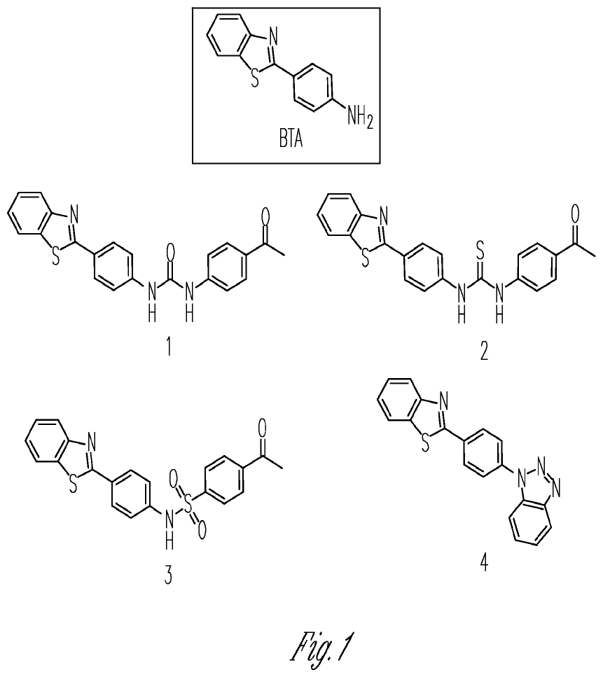

Compounds encompassed by the formulae described herein, such as the compounds shown in FIG. 1 comprising a urea (1), thiourea (2), sulfonamide (3), triazole (4) (FIG. 1), and triazine (Table 1) were synthesized and their anti-fibrillary effect on islet amyloid polypeptide (IAPP), amyloid-beta (Aβ1-40, Aβ1-42), α-synuclein (α-syn), and transthyretin (TTR81-127, TTR101-125) were explored using ThT fluorescence assays. The ant-fibrillary and anti-oligomer effect compounds with superior activity (e.g., high maximum thioflavin T (ThT) intensity in percentage at 100 μM) were further explored with α-synuclein (α-syn) and tau isoform 2N4R (tau 2N4R) by transmission electron microscopy (TEM) and photo-induced cross-linking of unmodified proteins (PICUP), respectively.

The above compounds, and pharmaceutically acceptable salts and solvates (e.g., hydrates) thereof, can be synthesized in accordance with methods known in the art and exemplified herein. See, e.g., Example 1.

The term “electron-withdrawing group” as the term is used herein refers to a functional group or electronegative atom that draws electron density away from an atom to which it is bonded either inductively and/or through resonance, whichever is more dominant (e.g., a functional group or atom may be electron donating through resonance but may overall be electron withdrawing inductively) and tends to stabilize anions or electron rich moieties. The electron withdrawing effect is typically transmitted inductively, albeit in attenuated form, to other atoms attached to the bonded atom that has been made electron deficient by the electron withdrawing group (EWG) thus affecting the electrophilicity of a more remote reactive center.

Exemplary electron withdrawing groups include, but are not limited to —NC(O)R″, —NSO₂R″, —C(O)R″, —CN, —NO₂, —CX¹₃, X¹, —C(O)OR″, —C(O)NH₂, —C(O)NR″₂, —C(O)R″, —C(O)X, —S(O)₂R″, —S(O)₂OR″, —SO₃H₂, —S(O)₂NH₂, —S(O)₂NR″₂, —PO₃H₂, —P(O)(OR″)₂, —NO, —NH₂, —NR₂″, —N(R″)₃⁺, and salts thereof, wherein X¹ is —F, —Br, —Cl, or —I, and each R″ is, at each occurrence, independently selected from H, C₁₋₆ alkyl and aryl (e.g., phenyl). Exemplary EWGs can also include aryl groups (e.g., phenyl) depending on substitution and certain heteroaryl groups (e.g., pyridine). Thus, the term “electron withdrawing groups” also includes aryls or heteroaryls that are further substituted with electron withdrawing groups. Typically, electron withdrawing groups are —C(O), —CN, —NO₂, —CX₃, and —X, wherein X is halogen. Depending on its substituents, an optionally substituted alkyl moiety may also be an electron withdrawing group.

“Alkyl” refers to a fully saturated cyclic or acyclic, branched or unbranched carbon chain moiety having the number of carbon atoms specified, or up to 30 carbon atoms if no specification is made. For example, an alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. A straight chain or branched chain alkyl can have 30 or fewer carbon atoms in its backbone (e.g., C₁-C₃₀ for straight chains, C₃-C₃₀ for branched chains), or or fewer. Alkyl groups may be substituted or unsubstituted.

“Alkylene” refers to an alkyl group having the specified number of carbons, for example from 2 to 12 carbon atoms, that contain two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moieties and may be optionally substituted with one or more substituents. For example, alkylene-aryl can be benzyl.

“Cycloalkyl” means mono- or bicyclic or bridged or spirocyclic, or polycyclic saturated carbocyclic rings, each having from 3 to 12 carbon atoms. In various aspects, cycloalkyls have from 3-10 carbon atoms in their ring structure, or 3-6 carbons in the ring structure. Cycloalkyl groups may be substituted or unsubstituted. Examples of cycloalkyl include cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms can range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety.

Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl but having one or more triple bonds in the moiety.

Unless the number of carbons is otherwise specified, “lower alkyl,” as used herein, means an alkyl group, as defined above, but having from one to ten carbons, or from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. A substituent designated herein as alkyl can be a lower alkyl.

“Heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

“Heteroarylalkyl” refers to alkyl groups as defined herein in which a hydrogen or carbon bond of an alkyl group is replaced with a bond to a heteroaryl group as defined herein.

“Aryl” includes 3- to 12-membered substituted or unsubstituted single-ring aromatic groups in which each atom of the ring is carbon (i.e., carbocyclic aryl) or where one or more atoms are heteroatoms (i.e., heteroaryl). In various aspects, aryl groups include 5- to 12-membered rings, or 6- to 10-membered rings. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carbocyclic aryl groups include benzene, naphthalene, phenanthrene, phenol, aniline, and the like. “Heteroaryl” groups include substituted or unsubstituted aromatic 3- to 12-membered ring structures, 5- to 12-membered rings, or 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic. Each instance of an aryl group may be independently optionally substituted, i.e., unsubstituted (an “unsubstituted aryl”) or substituted (a “substituted aryl”) with one or more substituents, e.g., for instance from 1 to 5 substituents, 1 to 4 substituents, 1 to 3 substituents, 1 to 2 substituents or just 1 substituent. The aromatic ring may be substituted at one or more ring positions with one or more substituents, such as halogen, azide, alkyl, aryl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. For example, the aryl group can be an unsubstituted C₅-C₁₂ aryl or the aryl group can be a substituted C₅-C₁₀ aryl. Aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

“Amine” refers to primary, secondary, and tertiary amines having, e.g., the formula N(group)₃ wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and RAN, wherein each R is independently selected, such as trialkylamines, dialkylarylamines, alkyldiarylamines, triarylamines, and the like. The term “amine” also includes ammonium ions as used herein.

“Amino” and “amino group” are used to refer to a substituent of the form —NH₂, —NHR, —NR₂, or —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, dialkylamino, and trialkylamino group.

The term “halo,” “halide,” or “halogen” means halogen and includes, for example, and without being limited thereto, fluoro (—F), chloro (—Cl), bromo (—Br), iodo (—I) and the like, in both radioactive and non-radioactive forms. Halo can be selected from the group consisting of fluoro, chloro and bromo.

“Haloalkyl” includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

“Nitro” means —NO₂; “sulfhydryl” means —SH; “hydroxy” or “hydroxyl” means —OH; “sulfonyl” means —SO₂—; “azido” means —N₃; “cyano” means —CN; “isocyanato” means —NCO; “thiocyanato” means —SCN; “isothiocyanato” means —NCS; and “cyanato” means —OCN.

“Alkoxy” refers to an oxygen atom connected to an alkyl group, including a cycloalkyl group, as defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The above compounds include isotopic variants and compounds in which one or more hydrogen atoms have been substituted with deuterium. The compounds may contain one or more chiral centers or may otherwise be capable of existing as multiple stereoisomers. In one embodiment, the compounds are not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds may include geometric centers, such as cis, trans isomers, diastereomers, enantiomers, and E and Z double bonds. In another embodiment, the compounds are not limited to any particular geometric isomer requirement, and that the compounds, and compositions, methods, uses, and medicaments that include them may be pure, or may be any of a variety of geometric isomer mixtures. Such mixtures of geometric isomers may include a single configuration at one or more double bonds and chiral carbons, while including mixtures of geometry at one or more other double bonds and chiral carbons.

The terms “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates.

The term “solvate” means a compound, or a salt thereof, that further includes a stoichiometric or non-stoichiometric amount of solvent bound by non-covalent intermolecular forces. Where the solvent is water, the solvate is a hydrate.

In each of the foregoing and following embodiments, it is also to be understood that the compounds described herein include and represent any and all crystalline forms, partially crystalline forms, and non-crystalline and/or amorphous forms of the compounds.

Further provided is a pharmaceutical composition comprising one or more of the above-described compounds, or pharmaceutically acceptable salts thereof, and a pharmaceutically acceptable carrier. The compounds can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington: The Science and Practice of Pharmacy, 23^{rd} edition, Oct. 30, 2020, Adeboye Adejare, ed.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The compounds described herein can be used to inhibit the aggregation of proteins prone to aggregate in a state of disease. The protein prone to aggregate can be islet amyloid polypeptide, amyloid-β, α-synuclein, tubulin associated unit (tau), or transthyretin. The tau can be tau isoform 2N4R or 1N4R. The disease can be AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis.

The compounds can be used to inhibit tau protein aggregation in tauopathies. Tauopathies are a group of disorders that result from abnormal tau phosphorylation, abnormal levels of tau, abnormal tau splicing, and mutations in the tau gene, for example. Neurodegenerative diseases have been classified based on this protein accumulation. Tauopathies encompass more than 20 clinicopathological conditions, including Alzheimer's disease (AD), which is the most common tauopathy. Other tauopathies include, but are not limited to, familial AD, primary age-related tauopathy (PART), Creutzfeldt-Jacob disease, dementia pugilistica, Gerstmann-Straussler-Scheinker disease (GSS), inclusion-body myositis, cortico-basal degeneration (CBD), Picks disease (PiD), progressive supranuclear palsy (also known as Steele, Richardson, and Olszewski disorder), Down syndrome, Parkinsonism with dementia, myotonic dystrophy, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, diffuse neurofibrillary tangles with calcification, frontotemporal dementia with Parkinsonism linked to chromosome 17 (FTDP-17), Haller-vorden-Spatz disease, multiple system atrophy (MSA), Niemann-Pick disease type C, pallido-ponto-nigral degeneration, progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD, Huntington's disease, and prion diseases with tangles. The majority of neurodegenerative diseases are characterized by the deposition of insoluble protein in cells of the neuromuscular system.

Still further provided is a method of inhibiting protein aggregation in a subject having, or at risk for, aggregation of a protein prone to aggregate in a state of disease, the method comprising administering to the subject one or more of the above-described compounds, or pharmaceutically acceptable salts thereof (e.g., in a pharmaceutical composition comprising one or more of the above-described compounds, or pharmaceutically acceptable salts thereof, and a pharmaceutically acceptable carrier), in an amount effective to inhibit protein aggregation. The protein prone to aggregate can be one or more of islet amyloid polypeptide, amyloid-β, α-synuclein, tubulin associated unit (tau), or transthyretin. The tau can be tau isoform 2N4R or 1N4R. The disease can be AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis. The subject can have, or be at risk for, Alzheimer's disease. The subject can have, or be at risk for, Parkinson's disease. Four BTA derivatives containing urea (1), thiourea (2), sulfonamide (3), triazole (4) (FIG. 1), and triazine (5-14) (Table 1) were synthesized. The anti-fibrillary effect of BTA and its derivatives on islet amyloid polypeptide (IAPP), amyloid-beta (Aβ1-40, Aβ1-42), α-synuclein (α-syn), and transthyretin (TTR81-127, TTR101-125) was explored using ThT fluorescence assays. The ant-fibrillary and anti-oligomer effect of the best compound was further explored with α-synuclein (α-syn) and tau isoform 2N4R (tau 2N4R) by transmission electron microscopy (TEM) and photo-induced cross-linking of unmodified proteins (PICUP), respectively.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

The disclosure also includes the following Statements:

Statement 1 relates to a compound comprising a benzisothiazole linked to an amide, a formamide, an amine, or a urea, or a pharmaceutically acceptable salt thereof, wherein at least one carbon on the benzyl ring of the benzisothiazole is substituted with a substituent selected from the group consisting of a C₁-C₆ alkyl, —NO₂, and a halo.

Statement 2 relates to the compound of Statement 1, or a pharmaceutically acceptable salt thereof, wherein the C₁-C₆ alkyl is methyl.

Statement 3 relates to the compound of Statement 1 or 2, or a pharmaceutically acceptable salt thereof, wherein the halo is F, Cl, or Br.

Statement 4 relates to the compound of Statement 1, wherein the compound is a compound of the formula:

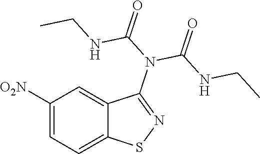 [SMILES: CCNC(=O)N(C(=O)NCC)c1nsc2ccc([N+](=O)[O-])cc12]

or a pharmaceutically acceptable salt thereof.

Statement 5 relates to the compound of Statement 1, wherein the compound is a compound of the formula:

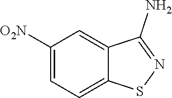 [SMILES: Nc1nsc2ccc([N+](=O)[O-])cc12]

or a pharmaceutically acceptable salt thereof.

Statement 6 relates to the compound of Statement 1, wherein the compound is a compound of the formula:

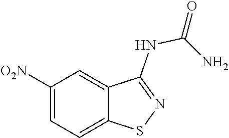 [SMILES: NC(=O)Nc1nsc2ccc([N+](=O)[O-])cc12]

or a pharmaceutically acceptable salt thereof.

Statement 7 relates to the compound of Statement 1, wherein the compound is a compound of the formula:

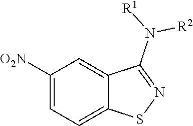

or a pharmaceutically acceptable salt thereof,
wherein:
each R₁ and R₂ is independently selected from hydrogen, alkyl, cycloalkyl, aryl, heteroaryl, —SO₂R, and —COR, wherein R₁ and R₂ are each, independently, optionally substituted with an alkyl, a nitro, a cyano, a halo, an amino, a hydroxy, an alkoxy, an aryl, or a heteroaryl, and wherein R is an alkyl, an alkenyl, an alkynyl, a cycloalkyl, a heterocycloalkyl, an aryl, or a heteroaryl, and wherein R is optionally substituted with a halo, an amino, a nitro, a cyano, a hydroxy, an alkoxy, an aryl, or a heteroaryl group.

Statement 8 relates to the compound of Statement 7, wherein the compound is a compound of the formula:

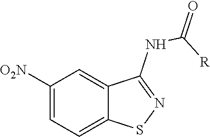

or a pharmaceutically acceptable salt thereof,
wherein:
R is an alkyl, an alkenyl, an alkynyl, a cycloalkyl, a heterocycloalkyl, an aryl, or a heteroaryl, and wherein R is optionally substituted with a halo, an amino, a nitro, a cyano, a hydroxy, an alkoxy, an aryl, or a heteroaryl group.

Statement 9 relates to the compound of Statement 8, or a pharmaceutically acceptable salt thereof,
wherein R is —CH₂—Cl.

Statement 10 relates to the compound of Statement 7, or a pharmaceutically acceptable salt thereof,
wherein R₁ and R₂ are hydrogen.

Statement 11 relates to a compound of the formula:

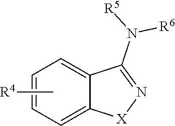

or a pharmaceutically acceptable salt thereof;
wherein:
X is O, NR³, or S, wherein R³ is H or alkyl;
R⁴ is an electron withdrawing group; and
each R⁵ and R⁶ is independently selected from hydrogen, alkyl, cycloalkyl, aryl, heteroaryl, —SO₂R, —C(O)NHR, and —C(NR⁷)NHR, wherein R⁵ and R⁶ can each be independently optionally substituted with an alkyl, a nitro, a cyano, a halo, an amino, a hydroxy, an alkoxy, an aryl, or a heteroaryl, and R is an alkyl, an alkenyl, an alkynyl, a cycloalkyl, a heterocycloalkyl, an aryl, or a heteroaryl, and wherein R can be optionally substituted with a halo, an amino, a nitro, a cyano, a hydroxy, an alkoxy, an aryl, or a heteroaryl group.

Statement 12 relates to the compound of Statement 11, wherein the compound is a compound of the formula:

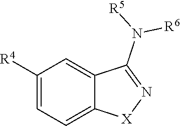

or a pharmaceutically acceptable salt thereof.

Statement 13 relates to a compound of the formula:

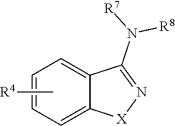

or a pharmaceutically acceptable salt thereof;
wherein:
X is O, NR³, or S, wherein R³ is H or alkyl;
R⁴ is an electron withdrawing group; and
each R⁷ and R⁶ is independently selected from H, —C(O)NHR, alkylaminoalkyl, aminoalkyl, arylamino, heteroarylamino, alkyl substituted with at least one group comprising a hydrogen atom capable of hydrogen bonding with at least one hydrogen bonding acceptor, aryl substituted with at least one group comprising a hydrogen atom capable of hydrogen bonding with a hydrogen bonding acceptor, heteroaryl substituted with at least one group comprising a hydrogen atom capable of hydrogen bonding with a hydrogen bonding acceptor, and heteroaryl comprising at least one hydrogen atom capable of hydrogen bonding with a hydrogen bonding acceptor, each of which can independently optionally be substituted with an alkyl, a nitro, a cyano, a halo, an amino, a hydroxy, an alkoxy, an aryl, or a heteroaryl, and R is an alkyl, an alkenyl, an alkynyl, a cycloalkyl, a heterocycloalkyl, an aryl, or a heteroaryl and wherein R can be optionally substituted with a halo, an amino, a nitro, a cyano, a hydroxy, an alkoxy, an aryl, or a heteroaryl group.

Statement 14 relates to the compound of Statement 13, wherein the compound is a compound of the formula:

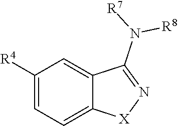

or a pharmaceutically acceptable salt thereof.

Statement 15 relates to the compound of Statement 13, or a pharmaceutically acceptable salt thereof, wherein R⁷ is H and R⁸ is selected from H, —C(O)NHR, alkylaminoalkyl, aminoalkyl, arylamino, and heteroarylamino, each of which can be optionally substituted with a halo, an amino, a nitro, a cyano, a hydroxy, an alkoxy, an aryl, or a heteroaryl group.

Statement 16 relates to the compound of Statement 13, or a pharmaceutically acceptable salt thereof, wherein the group comprising a hydrogen atom capable of hydrogen bonding with a hydrogen bonding acceptor is —C(O)OH, —C(O)NHR, or —OH.

Statement 17 relates to the compound of Statement 13, or a pharmaceutically acceptable salt thereof, wherein the heteroaryl comprising at least one hydrogen atom capable of hydrogen bonding with a hydrogen bonding acceptor is selected from:

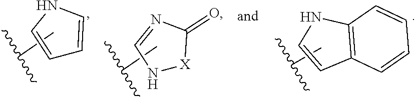

Statement 18 relates to the compound of Statements 11-13, or a pharmaceutically acceptable salt thereof, wherein the electron withdrawing group is selected from —NC(O)R″, —NSO₂R″, —C(O)R″, —CN, —NO₂, —CX¹₃, X¹, —C(O)OR″,
—C(O)NH₂, —C(O)NR″₂, —C(O)R″, —C(O)X, —S(O)₂R″, —S(O)₂OR″, —SO₃H₂, —S(O)₂NH₂, —S(O)₂NR″₂, —PO₃H₂, —P(O)(OR)₂, —NO, —NH₂, —NR₂″, —N(R″)₃⁺, and salts thereof, wherein X¹ is —F, —Br, —Cl, or —I, and each R″ is, at each occurrence, independently selected from H, C₁₋₆ alkyl and aryl.

Statement 19 relates to the compound of Statements 11-13, or a pharmaceutically acceptable salt thereof, wherein the electron withdrawing group is selected from halo, —NO₂, —CN, —CF₃, or —C(O)R″, wherein R″ can be H, C₁₋₆ alkyl and aryl.

Statement 20 relates to a pharmaceutical composition comprising at least one compound of any one of Statements 1-19, or a pharmaceutically acceptable salt thereof, and a pharmaceutically acceptable carrier.

Statement 21 relates to a method of inhibiting protein aggregation in a subject having, or at risk for, aggregation of a protein prone to aggregate in a state of disease, which method comprises administering to the subject a compound of Statements 1-19 or the composition of Statement 20 in an amount effective to inhibit protein aggregation, whereupon protein aggregation is inhibited in the subject having, or at risk for, aggregation of a protein prone to aggregate in a state of disease.

Statement 22 relates to the method of Statement 21, wherein the protein prone to aggregate is islet amyloid polypeptide, amyloid-0, α-synuclein, tubulin associated unit (tau), or transthyretin.

Statement 23 relates to the method of Statement 22, wherein the tau is tau isoform 2N4R or 1N4R or 0N4R, 2N3R or 1N3R or 0N3R with or without post-translational changes.

Statement 24 relates to the method of Statement 21 or 22, wherein the disease is AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis.

Statement 25 relates to the method of any one of Statements 21-24, wherein the subject has, or is at risk for, Alzheimer's disease.

Statement 26 relates to the method of any one of Statements 21-24, wherein the subject has, or is at risk for, Parkinson's disease.

### EXAMPLES

The disclosure can be better understood by reference to the following examples which are offered by way of illustration. The disclosure is not limited to the examples given herein.

### Chemical and Peptide Sources.

Hexafluoroisopropanol (HFIP), DMSO, and thioflavin-T (ThT) were purchased from Alfa Aesar (Ward Hill, MA). 4-(2-benzothiazolyl)aniline was obtained from Sigma Aldrich (Burlington, MA). TTR fragment 81-127 was obtained from AnaSpec (Fremont, CA). TTR fragments 1-25, 26-50, 51-75, 76-100, 81-105, 101-125, and 101-125 were obtained from GenScript (Piscataway, NJ). α-syn, Aβ₁₋₄₀, Aβ₁₋₄₂ were procured from rPeptide (WatKinsville, GA). Human islet amyloid polypeptide (IAPP) was procured from AnaSpec (Freemont, CA).

The bacterial expression plasmid consisting of the vector pRK172 carrying a cDNA encoding the human Tau 2N4R isoform was obtained from Dr. David Eliezer (Weill Cornell Medicine, New York, NJ). For protein expression, E. coli BL21(DE3) cells were transformed with the plasmid and grown in LB media supplemented with ampicillin (100 μg/mL). Protein over-expression was induced by the addition of 1 mM IPTG for 4 hours at 37° C., and cells were pelleted by centrifugation at 6,000 g for 15 minutes at 4° C. The cells were resuspended in lysis buffer (20 mM MES, 400 mM NaCl, 0.2 mM MgCl₂, 1 mM EGTA, protease inhibitor cocktail (P8340, Sigma Aldrich), 0.25 mg/mL lysozyme, and 1 μg/mL DNase 1, pH 6.8) and lysed by a French press cell disruptor at 4° C., after which the lysate was boiled for 20 minutes. Denatured proteins were pelleted by centrifugation at 30,000 g for 30 minutes at 4° C., and the supernatant was dialyzed overnight against cation exchange buffer (20 mM MES, 50 mM NaCl, 1 mM MgCl₂, 1 mM EGTA, 2 mM DTT, 0.1 mM PMSF, pH 6.8). The dialysate was loaded onto a HiPrep SP HP column, and proteins were eluted with a linear gradient ranging from 50 mM to 1 M NaCl. Fractions containing tau isoform 2N4R were pooled, and the resulting protein solution was dialyzed against PBS (pH 7.4) and stored at −80° C.

### Example 1

### Synthesis

All chemicals were obtained from conventional commercial sources and used without further purification unless otherwise noted. Compound 4 was synthesized using the procedure reported in Med. Chem. Commun., 2016, 7, 1151.³⁴ The ¹H NMR and ¹³C NMR chemical shifts are reported in parts per million relative to tetramethylsilane or the residual solvent protons.

1-(4-acetylphenyl)-3-[4-(1,3-benzothiazol-2-yl)phenyl]urea (1). To a stirred solution of 4-(benzo[d]thiazol-2-yl)aniline (1 eq) in anhydrous dichloromethane (10 mL) under nitrogen atmosphere was added 4-acetylphenyl isocyanate (1.1 eq) dropwise. The reaction was stirred at room temperature for 24 hrs until a precipitate was formed. On completion of the reaction monitored by TLC, the precipitate was filtered, washed thrice with ether, and dried in vacuo to obtain the desired product (compound 1,122 mg) with a yield of 71%. ¹H NMR (500 MHz, DMSO) δ 9.21 (d, J=4.9 Hz, 2H), 8.19-7.83 (m, 6H), 7.72-7.31 (m, 6H), 2.51 (s, 3H). ¹³C NMR (126 MHz, DMSO) δ 196.8, 167.5, 154.1, 152.4, 144.5, 142.8, 134.7, 131.1, 130.1, 128.6, 127.0, 127.0, 125.6, 123.0, 122.7, 118.9, 117.8, 26.8. HRMS-ESI (m/z): [M+H]⁺ calcd for C₂₂H₁₈N₃O₂S, 387.1120, found [M+H]⁺ 388.1123.

1-(4-acetylphenyl)-3-[4-(1,3-benzothiazol-2-yl)phenyl]thiourea (2). To a stirred solution of 4-(benzo[d]thiazol-2-yl)aniline (1 eq) in anhydrous tetrahydrofurane (30 mL) under nitrogen atmosphere was added 4-acetylphenyl isothiocyanate (1.1 eq) dropwise. The reaction was refluxed for 24 hrs. On completion of the reaction monitored by TLC, the precipitate was filtered, washed thrice with ether, and dried in vacuo to obtain the desired product (compound 2, 135 mg) with a yield of 76%. ¹H NMR (500 MHz, DMSO) δ 10.36 (d, J=13.3 Hz, 2H), 8.25-7.86 (m, 6H), 7.83-7.30 (m, 6H), 2.53 (s, 3H). ¹³C NMR (126 MHz, DMSO) δ 197.1, 179.5, 167.3, 154.1, 144.4, 142.7, 134.8, 132.8, 129.4, 129.0, 128.1, 127.1, 125.8, 123.6, 123.1, 122.8, 122.3, 27.0. HRMS-ESI (m/z): [M+H]⁺ calcd for C₂₂H₁₇N₃NaOS₂, 426.0711, found [M+Na]⁺ 426.0711.

4-acetyl-N-[4-(1,3-benzothiazol-2-yl)phenyl]benzene-1-sulfonamide (3). To a solution of the 2-(4-aminophenyl)benzothiazole (200 mg, 0.88 mmol) in pyridine (2 mL) was added 4-acetylbenzenesulfonyl chloride (1.5 eq). The reaction mixture was heated under reflux for 10 min and then was cooled, after which water (5 mL) was added. The precipitate formed was collected by filtration, washed with water, and dried in vacuo to obtain the desired product (compound 3, 355 mg) with a yield of 91%. ¹H NMR (500 MHz, DMSO) δ 10.94 (s, 1H), 8.14-8.04 (m, 3H), 8.03-7.89 (m, 5H), 7.49 (ddd, J=8.3, 7.2, 1.3 Hz, 1H), 7.41 (ddd, J=8.3, 7.2, 1.2 Hz, 1H), 7.35-7.19 (m, 2H), 2.56 (s, 3H). ¹³C NMR (126 MHz, DMSO) δ 197.7, 167.0, 154.0, 143.3, 140.7, 140.4, 134.8, 129.7, 129.0. 127.6, 127.1, 125.9. 123.1, 122.8, 120.1, 27.5. HRMS-ESI (m/z): [M+H]⁺ calcd for C₂₁H₁₇N₂O₃S₂, 409.0681, found [M+H]⁺ 409.0702.

1-[4-(1,3-benzothiazol-2-yl)phenyl]-1H-1,2,3-benzotriazole (4). Compound 4 was synthesized using the procedure reported as published previously.³⁴ Amount: 128 mg, 88%. ¹H NMR (500 MHz, DMSO) δ 8.37 (d, J=8.3 Hz, 2H), 8.20 (dd, J=16.4, 8.2 Hz, 2H), 8.11 (t, J=7.7 Hz, 3H), 8.06 (d, J=8.4 Hz, 1H), 7.76-7.66 (m, 1H), 7.56 (dt, J=12.5, 7.5 Hz, 2H), 7.49 (t, J=7.6 Hz, 1H). ¹³C NMR (126 MHz, DMSO) δ 166.4, 154.1, 146.4, 138.9, 135.2, 133.2, 132.0, 129.5, 129.4, 127.3, 126.3, 125.5, 123.6, 123.0, 120.4, 111.7.

N-[4-(1,3-benzothiazol-2-yl)phenyl]-4,6-dichloro-1,3,5-triazin-2-amine (5). To a stirred solution of amine (1 eq) in DCM (10 mL) was added cyanuric chloride (1 eq) and triethylamine (1 eq). Reaction was stirred for 8-12 h at room temperature. The precipitate obtained was filtered and washed thrice with diethyl ether to obtain the desired product (compound 5, 76 mg) with the yield of 26%. ¹H NMR (500 MHz, DMSO) δ 11.42 (s, 1H), 8.16-8.06 (m, 3H), 8.02 (ddd, J=8.1, 1.2, 0.6 Hz, 1H), 7.80 (d, J=8.8 Hz, 2H), 7.52 (ddd, J=8.3, 7.2, 1.3 Hz, 1H), 7.43 (ddd, J=8.3, 7.2, 1.2 Hz, 1H). ¹³C NMR (126 MHz, DMSO) δ 170.2, 167.1, 164.2, 154.1, 140.3, 134.9, 129.4, 128.5, 127.1, 125.9, 123.2, 122.8, 121.9. HRMS-ESI (m/z): m/z calcd for C₁₆H₉Cl₂N₅S, 372.9956, found 372.0515.

N-(4,6-dichloro-1,3,5-triazin-2-yl)-5-nitro-1,2-benzothiazol-3-amine (6). Cyanuric chloride (138 mg, 0.75 mmol, 1.0 equiv.) was dissolved in THF (6.0 mL). The reaction mixture was cooled to 0° C. on an ice bath and treated with DIPEA (117 μL, 0.67 mmol, 0.9 equiv.) at 0° C. After 5 min, reaction mixture was treated with 3-amino-5-nitrobenzisothiazole (146 mg, 0.75 mmol, 1.0 equiv.) and stirred at 0° C. for 30 min. Then the ice bath was removed, and the reaction mixture was stirred at room temperature. The reaction progression was monitored by TLC (hexane:ethyl acetate; 7:3). After 30 min, the crude was purified by FCC (hexane:ethyl acetate; 8:2) to obtain the clean product compound 6 (66 mg) with the yield of 26%. Yellow color solid. ¹H NMR (500 MHz, DMSO) δ 11.91 (s, 1H), 8.80 (d, J=2.7 Hz, 1H), 8.57 (dd, J=9.1, 2.7 Hz, 1H), 7.92 (d, J=9.0 Hz, 1H). ¹³C NMR (126 MHz, DMSO) δ 170.3, 165.5, 145.4, 144.7, 129.8, 129.4, 127.8, 115.4, 109.2. IR (solid) v/cm⁻¹: 3303, 3118, 3084, 2233, 1571, 1535, 1504.

N-(4-chloro-1,3,5-triazin-2-yl)-5-nitro-1,2-benzothiazol-3-amine (7). 2,4-Dichloro-1,3,5-triazine (195 mg, 1.00 mmol, 1.0 equiv.) was dissolved in THF (10.0 mL). The reaction mixture was cooled to 0° C. on an ice bath. 3-amino-5-nitrobenzisothiazole (150 mg, 1.00 mmol, 1.0 equiv.) was added and the mixture was stirred at 0° C. for 30 min. Then the ice bath was removed, and the reaction mixture was stirred at room temperature for 48 hrs. The reaction progression was monitored by TLC (hexane:ethyl acetate; 7:3), and the crude was purified by FCC (hexane:ethyl acetate; 8:2) to obtain the clean product compound 7 (66 mg) with the yield of 26%. Yellow color solid. ¹H NMR (500 MHz, CDCl₃) δ 8.88 (d, J=9.4 Hz, 1H), 8.78 (s, 1H), 8.62-8.44 (m, 2H), 8.14 (s, 1H). ¹³C NMR (126 MHz, CDCl₃) δ 171.5, 168.0, 163.9, 144.5. 142.9, 129.3, 128.5, 121.2, 114.1, 103.0. IR (solid) V/cm⁻¹: 3221, 3063, 2242, 1616, 1567, 1545, 1494, 1397.

N-(5-nitro-1,2-benzothiazol-3-yl)-2-phenylacetamide (8). 3-amino-5-nitro-benzothiazole (150 mg, 0.77 mmol) was dissolved in 3 mL pyridine. Then, phenylacetyl chloride was slowly added (0.11 mL, 0.85 mmol), and the reaction mixture was stirred for 4 h at room temperature. The rection mixture was poured into ice and neutralized by 2N HCl, then extracted with dichloromethane (15 mL×3) and dried over anhydrous magnesium sulfate. The crude product was purified by column chromatography (hexane:ethyl acetate, 4:1 v/v) to obtain the compound 8 as a pale-yellow powder (192 mg, 81%). ¹H NMR (500 MHz, DMSO) δ 10.78 (s, 1H), 8.70 (d, J=2.7 Hz, 1H), 8.46 (dd, J=9.2, 2.7 Hz, 1H), 7.98 (d, J=9.2 Hz, 1H), 7.37-7.31 (m, 4H), 7.28-7.23 (m, 1H), 3.81 (s, 2H). ¹³C NMR (126 MHz, DMSO) δ 170.7, 146.4, 143.6, 135.5, 129.8, 129.8. 129.4, 128.9, 127.3, 125.0, 115.6, 106.2, 43.1. IR (solid) v/cm⁻¹: 3187, 3010, 2228, 1682, 1580, 1505, 1405.

N-(5-nitro-1,2-benzothiazol-3-yl)-2-phenylacetamide (9). 3-amino-5-nitrobenzisothiazole (98 mg, 0.50 mmol, 1.0 equiv.) was dissolved in THF (15.0 mL). The reaction mixture was cooled to 0° C. on an ice bath. The reaction mixture was charged with 2-thiopheneacetyl chloride (64 μL, 0.52 mmol, 1.05 equiv.) and stirred for 30 min at 0° C. After 30 in, it was gradually warmed to room temperature and stirred at room temperature until all the amine starting materials were consumed, followed by TLC (hexane:ethyl acetate; 7:3). Then, the reaction mixture was directly loaded to a column and purified by FCC (hexane:ethyl acetate; 7:3). The clean product (compound 9, 20 mg) was obtained with the yield of 13%. Yellow color solid. ¹H NMR (500 MHz, DMSO) δ 9.38 (s, 1H), 8.10 (dd. J=9.6, 2.4 Hz, 1H), 7.69 (d, J=9.6 Hz, 1H), 7.45 (d, J=5.1 Hz, 1H), 7.04 (dd, J=30.3, 4.0 Hz, 2H), 4.28 (s, 2H). ¹³C NMR (126 MHz, DMSO) δ 169.8, 163.8, 158.1, 142.0, 135.6, 127.7, 127.4, 126.2, 122.6, 122.4, 120.3, 119.8, 35.9. IR (solid) v/cm⁻¹: 3280, 3090, 1686, 1603, 1527, 1495, 1418, 1316.

N-(5-nitro-1,2-benzothiazol-3-yl)acetamide (10). 3-amino-5-nitrobenzisothiazole (156 mg, 0.80 mmol, 1.0 equiv.) was dissolved in DMF (2 mL) at room temperature. Then reaction mixture was cooled to 0° C. on an ice bath. At 0° C., acetyl chloride (57 μL, 0.80 mmol, 1.0 equiv.) was added. The reaction mixture was stirred at for 30 min at 0° C. and then gradually warmed to room temperature. The mixture was stirred at room temperature until all amine starting material was consumed as monitored by TLC (hexane:ethyl acetate; 7:3). The crude mixture was evaporated by vacuum and purified by FCC (hexane:ethyl acetate; 9:1 to 7:3) to obtain pure product, compound 10 (12 mg), with the yield of 5%. R_{f}: 0.3 (Hexanes:EtOAc; 7:3). Yellow color solid. ¹H NMR (500 MHz, CDCl₃) δ 9.25 (dd, J=2.6, 0.5 Hz, 1H), 8.90 (dd, J=9.4, 0.6 Hz, 1H), 8.58 (dd, J=9.4, 2.5 Hz, 1H), 2.71 (s, 3H), 2.66 (s, 3H). ¹³C NMR (126 MHz, DMSO) δ 170.0, 164.0, 158.0, 141.9, 122.5, 122.3, 120.4, 119.5, 22.6. IR (solid) v/cm⁴¹: 3284, 1690, 1603, 1518, 1492, 1318, 1239.

2-chloro-N-(5-nitro-1,2-benzothiazol-3-yl)acetamide (11). 3-amino-5-nitro-benzothiazole (200 mg, 1.02 mmol) was dissolved in 2 mL DMF at 0° C. Anhydrous potassium carbonate (213 mg, 1.54 mmol) was then added, and the reaction mixture was stirred for 30 minutes. Chloroacetyl chloride (0.16 mL, 2.04 mmol) was then added in small portions, and the reaction was stirred overnight at room temperature. The crude product was purified by column chromatography (hexane:ethyl acetate, 5:1 v/v) to obtain the compound 11 as yellow powder (211 mg, 76%). ¹H NMR (500 MHz, DMSO) δ 9.28 (d, J=2.4 Hz, 1H), 8.07 (dd, J=9.6, 2.4 Hz, 1H), 7.93 (s, 1H), 7.68 (d, J=9.6 Hz, 1H), 4.64 (s, 2H). ¹³C NMR (126 MHz, DMSO) δ 166.4, 163.2, 162.8, 158.1, 142.2, 122.6, 122.4, 120.06, 120.0, 42.4. IR (solid) v/cm⁴: 3105, 2838, 1710, 1657, 1604, 1572, 1536.

N-methanesulfonyl-N-(5-nitro-1,2-benzothiazol-3-yl)methanesulfonamide (12). 3-amino-5-nitro-benzothiazole (200 mg, 1.02 mmol) was dissolved in 3 mL pyridine. Methanesulfonyl chloride (0.15 mL, 2.04 mmol) was added gradually, and the reaction mixture was stirred overnight at room temperature. The rection mixture was poured into ice and neutralized by 2N HCl, then extracted with dichloromethane (15 mL×3) and dried over anhydrous magnesium sulfate. The crude product was purified by column chromatography (hexane:ethyl acetate, 5:1 v/v) to obtain the compound as a yellow powder (265 mg, 79%). ¹H NMR (500 MHz, DMSO) δ 8.96 (s, 1H), 8.61 (d, J=8.2 Hz, 1H), 8.17 (d, J=8.3 Hz, 1H), 3.66 (s, 6H). ¹³C NMR (126 MHz, DMSO) δ 148.84, 140.9, 134.7, 129.9, 129.8, 116.9, 115.4, 44.2. IR (solid) v/cm⁻¹: 3046, 2242, 1612, 1528, 1351, 1162.

N-ethyl-1-[(ethylcarbamoyl)(5-nitro-1,2-benzothiazol-3-yl)amino]formamide (13). 3-amino-5-nitrobenzisothiazole (131 mg, 0.80 mmol, 1.0 equiv.) was dissolved in THF (10.0 mL). The reaction mixture was charged with ethyl isocyanate (126 μL, 1.6 mmol, 2.00 equiv.) and stirred, followed by TLC (hexane; ethyl acetate: 7:3) until all the amine starting material was consumed. Then, the reaction mixture was diluted with hexane (15 mL), and the resultant precipitate was filtered and washed with (hexane; Et₂O:1:1). The product compound 13 (159 mg) was obtained with the yield of 59%. Yellow color solid. ¹H NMR (500 MHz, DMSO) δ 8.74 (d, J=2.5 Hz, 1H), 8.66 (s, 1H), 8.48-8.31 (m, 2H), 8.01 (s, 1H), 3.29-3.13 (m, 4H), 1.29-0.97 (m, 6H). ¹³C NMR (126 MHz, DMSO) δ 170.6, 164.9, 152.2, 149.0, 141.7, 128.6, 122.8, 119.9, 117.9, 36.0, 35.9, 15.2, 15.0.

### Example 2

### Analysis

Thioflavin-T (ThT) fluorescence assays. Thioflavin-T (ThT) fluorescence assays were used to monitor fibril formation of recombinant Ap (fragments 1-40 and 1-42), recombinant α-syn, recombinant tau isoform 2N4R, synthetic IAPP, and synthetic TTR peptides treated with BTA and its derivatives. The IAPP ThT assay was performed in 10 mM PBS (pH 7.4) at a final concentration of 10 μM for both IAPP and ThT as published previously. Aβ fragments 1-40 and 1-42 were tested at 7 μM in 10 mM of PBS using ThT at a final concentration of 20 μM. Kinetics of α-syn fibrillization were performed as published previously.³⁶⁻³⁷ TTR fragment kinetics of fibril formation were assessed at 10 μM in 100 mM sodium acetate buffer supplemented with 100 mM KCl and 1 mM ethylenediaminetetraacetic acid (EDTA) (pH 4) with 20 μM ThT. The fluorescence emission experiments were performed with the excitation and emission wavelengths set at 440 and 485 nm, respectively, with a Synergy HT multi-mode microplate reader (BioTek, Winooski, VT). Samples were measured in three replicates, and the experiments were repeated three times using at least two different stock solutions. For each time point, arbitrary units of fluorescence were calculated from the mean values normalized against the maximum value in each completed assay. The lag time for each condition was calculated as previously described. All results contained in histograms were presented as mean±SEM. Data were analyzed by the one-way analysis of variance with Dunnett's multiple comparisons between controls and compounds. Differences were considered statistically significant at p<0.05.

Compounds with the highest anti-fibrillary activity were tested at 3.125, 6.25, 12.5, 25, 50, and 100 μM to obtain dose-response curves with prepared α-syn and 2N4R tau at 6.25 μM and 6 μM, respectively. Compounds were tested with α-syn using a previously published procedure. Concerning the tau (isoform 2N4R) kinetics of fibril formation, measurements of ThT fluorescence were performed with a solution of the protein diluted to a final concentration of 6 μM in PBS (pH 7.4) supplemented with 1.5 μM heparin, 20 μM ThT, 2.5 mM DTT, and 100 μM compound. Aliquots of the diluted protein solution (100 μL each) were pipetted into the wells of a 96-well plate, and a Teflon ball was added to each well. The plate was incubated at 37° C. with constant shaking at 1,000 rpm in a Tecan Spark plate reader. ThT fluorescence was measured every 15 minutes with excitation and emission wavelengths of 440 nm and 480 nm, and the data were plotted using GraphPad Prism.

Transmission electron microscopy (TEM). After performing analysis with ThT fluorescence assay, TEM was utilized to detect fibril formation. A volume of 10 μL was applied to a 400-mesh Formvar-carbon-coated copper grid (Electron Microscopy Sciences, Hatfield, PA). The grids were incubated for 1 minute and washed three times with distilled water. They were carefully air-dried and incubated for 1 minute in a fresh solution of 1% uranyl acetate. Samples were air-dried and observed using a transmission microscope. Visualization of the grids was performed with TEM (JEOL 1400 Flash, Japan). Acquisition of pictures was performed with settings of accelerating voltage of 100 kV and magnification of 25 k and/or 40 k.

Photo-induced Cross-linking of Unmodified Proteins (PICUP) Assay. To induce oligomerization by cross-linking, α-syn (from Rpeptide, LLC) and tau isoform 2N4R were diluted in 10 mM phosphate buffer (pH 7.4) to reach a final concentration of 10 μM. Different compounds were added to the protein solution at a final concentration of 50 μM, resulting in a molar ratio of 1:5. To confirm the gradual effect of the compounds on the inhibition of α-syn oligomerization, the compounds were tested at final concentrations of 3.125, 6.25, 12.5, 25, and 50 μM. The controls consisted of samples without light exposition, without Ru(bpy) or ammonium persulfate, and without compound (i.e., 0.125% DMSO). The cross-linking reaction was initiated by the addition of 2 μL of Ru(bpy) (300 μM final concentration) and 2 μL ammonium persulfate (6 mM final concentration). Samples were irradiated immediately. Light exposure was of a one-second duration for α-syn and a three-second duration for tau isoform 2N4R, with a 53 W (120 V) incandescent lamp installed in a homemade dark-box. Each tube contained a final volume of 20 μL. After irradiation, 8.3 μL of Laemmli loading buffer containing 15% β-mercaptoethanol were immediately added to the solution, followed by incubation at 95° C. for 10 minutes. The cross-linked samples were separated on a 16% SDS-PAGE gel and visualized by Coomassie blue staining.

α-Syn (or αS) inclusion-forming neuroblastoma cell experiment. Dox-inducible neuroblastoma cells M17D-TR/αS-3K::YFP have been used previously. 96-well plates were used with a cellular density of 30,000 cells per well. Compounds were added after 24 hours, and αS-3K::YFP transgene expression was induced 48 hours later. Induction was done by adding 1 μg per mL (final concentration) dox to culture media. Cells were incubated in the Incucyte Zoom 2000 platform (Essen Biosciences), and images (green, bright field) were taken continuously. Endpoint analysis of inclusion formation or growth was performed 48 hours after induction (96 hours after plating). The Incucyte processing definition ‘Inclusions’ was created as follows: Parameters, Fixed Threshold, Threshold (GCU) 50; Edge Split On, Edge Sensitivity 100; Cleanup, Hole Fill (μm²): 10, Adjust Size (pixels): 0; Filters, Area (μm²): max 50, Mean Intensity: min 60, Integrated Intensity: min 2000. Cell confluence was measured by the processing definition ‘Cells’: Parameters, Segmentation Adjustment 0.7; Cleanup, all parameters set to 0; Filters, Area (μm²): min 345.00. As described previously for the evaluation of protein expression by SDS-PAGE and Western Blotting in the LiCor system, αS-specific monoclonal antibody 4B12 (Thermofisher, Waltham, MA; 1:1000) and a polyclonal antibody to GAPDH (Sigma-Aldrich, St. Louis, MO, G9545; 1:5000) were used.

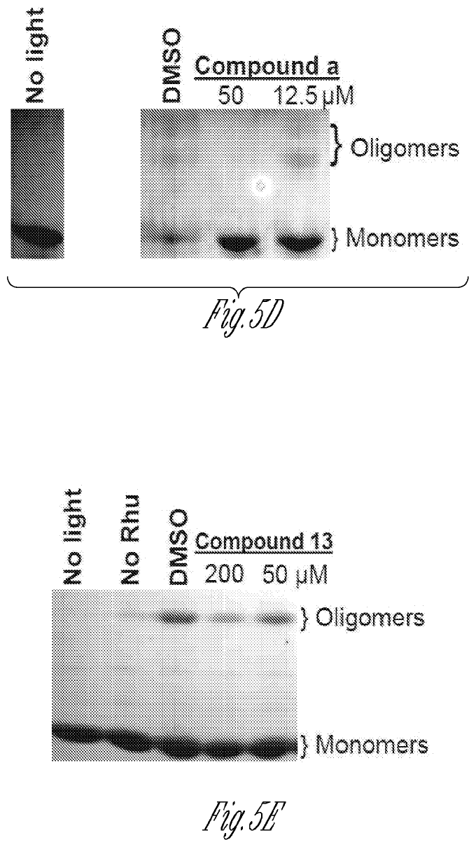

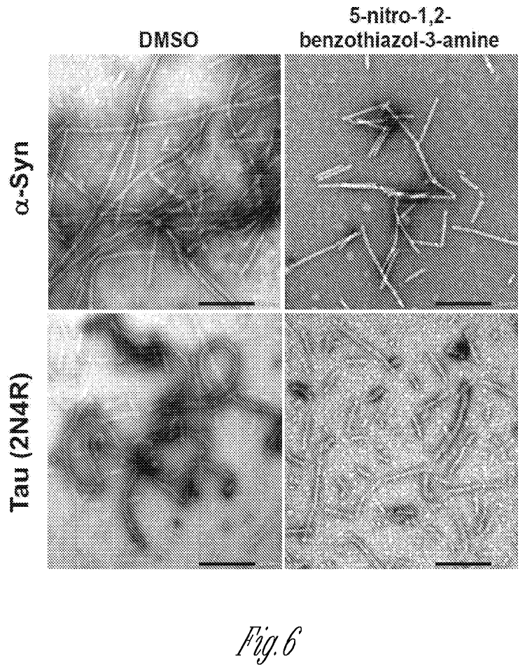

BTA is a general inhibitor of prone-to-aggregate proteins. To determine if BTA is a general or specific inhibitor of fibril formation, the kinetics of aggregation of α-syn, amyloid-beta fragments (Aβ₁₋₄₀ and Aβ₁₋₄₂), human IAPP, and TTR₈₁₋₁₂₇ in the presence and absence of BTA and resveratrol were assessed. IAPP, α-syn, and TTR₈₁₋₁₂₇ had arbitrary percent fluorescence under 40% for both resveratrol and BTA treatments. Transmission electron microscopy (TEM) was performed as follow up to confirm fibril alteration (FIG. 4). IAPP, α-synuclein, and TTR₈₁₋₁₂₇ were treated with either 100 μM resveratrol, 100 μM BTA, or 0.1% DMSO control at 37° C. for 1 day in PBS (IAPP, α-syn) or sodium acetate buffer (TTR₈₁₋₁₂₇) before analysis via TEM. Each sample was observed at 40 k magnification (IAPP and TTR₈₁₋₁₂₇) or 25 k magnification (α-syn). Magnification depended on the ability to observe the fibrils, and the best magnification was selected for each type with scale bars kept at 200 nm. Imaging via TEM confirmed reduced fibril formation in TTR fibrils treated with BTA and resveratrol (FIGS. 4G and 4J). The control treated samples featured the classic linear, branched fibril structure (FIGS. 4B, 4C, and 4D), while no such branching was observed on the copper grid containing samples treated with 100 μM BTA (FIGS. 4E, 4F, and 4G). Instead, the BTA treated TTR₈₁₋₁₂₇ fragment peptides were globular in shape (FIG. 4G).

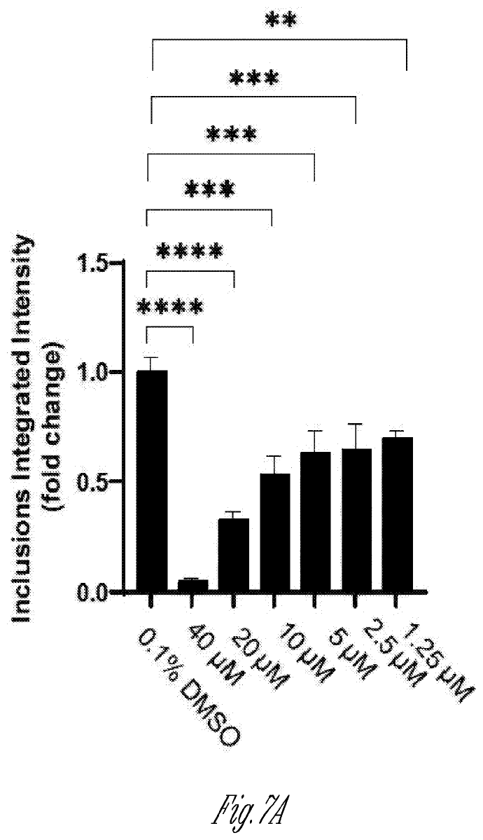

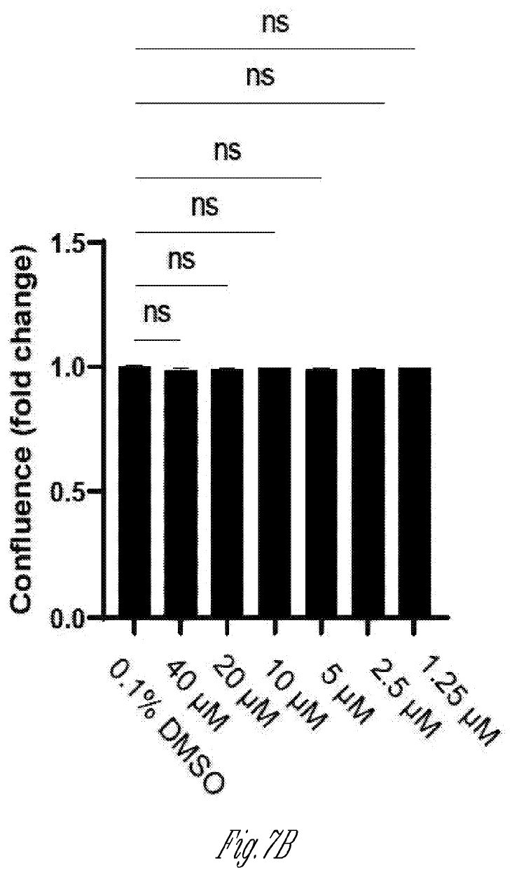

Truncated peptides, TTR₈₁₋₁₂₇ and TTR₁₀₁₋₁₂₅, were treated with BTA and four derivatives: compounds 1 (urea), 2 (thiourea), 3 (sulfonamide), 4 (triazole). ThT experiments were performed using the fragment peptides TTR₈₁₋₁₂₇ (FIG. 5A) and TTR₁₀₁₋₁₂₅ (FIG. 5B) to validate the anti-aggregation effect on these peptides. BTA and, to a lesser extent, compound 2 reduced fibril formation for both fragments at molar ratio 1:10 (FIGS. 5A and 5B). BTA and compounds 1-4 were tested at lower concentration using the TTR₁₀₁₋₁₂₅ fragment. Compound 2 was weak in inhibiting TTR₁₀₁₋₁₂₅ fibril formation at 50 μM (molar ratio 1:5) (FIG. 5C). BTA continued to reduce TTR₁₀₁₋₁₂₅ fibrils at 25 μM (molar ratio 1:2.5) (FIG. 5D). The anti-fibrillary effects of BTA and compound 2 were confirmed by TEM. The anti-aggregation effect of compound 2 was weaker in comparison to BTA and more compounds were designed exploring the triazine linker (Table 1). A significant improvement in anti-aggregation activity was observed with the triazine linker.

TABLE 1
Molecular structures of novel benzothiazole-linked derivatives and their
respective anti-fibrillary activity on α-synuclein (α-syn, 6.25 μM final
concentration) and transthyretin fragment peptide (TTR₈₁₋₁₂₇, 10 μM final
concentration) expressed as maximum thioflavin T (ThT) intensity in percentage
in which the compounds were tested at 100 μM. Photo-induced Cross-linking of
Unmodified Proteins (PICUP) oligomerization results are included with
compounds assayed at 50 μM. N.D .: not determined.

| | | ThT | ThT | |
|---|---|---|---|---|
| | | a-Syn | TTR₈₁₋₁₂₇ | PICUP |
| ID # | Structure | (%) | (%) | α-Syn |
| | Control (ThT: 0.25% DMSO; | 100.0 ± 14.2 | 100.1 ± 2.0 | [symbol] |
| | PICUP: 0.125% DMSO) | | | |
| A | 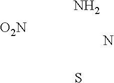 | 36.2 ± 3.1 | 54.7 ± 1.4 | [symbol] |
| | 5-nitro-1,2-benzothiazol-3-amine | | | |
| 5 | 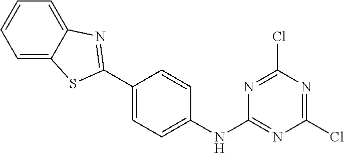 [SMILES: Clc1nc(Cl)nc(Nc2ccc(-c3nc4ccccc4s3)cc2)n1] | >100 | >100 | N.D. |
| 6 | 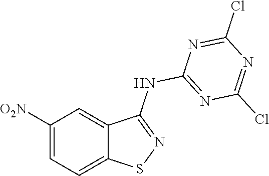 [SMILES: O=[N+]([O-])c1ccc2snc(Nc3nc(Cl)nc(Cl)n3)c2c1] | >100 | 82.6 ± 6.0 | [symbol] |
| 7 | 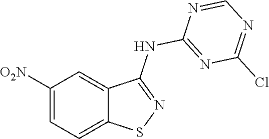 [SMILES: O=[N+]([O-])c1ccc2snc(Nc3ncnc(Cl)n3)c2c1] | >100 | >100 | [symbol] |
| 8 | 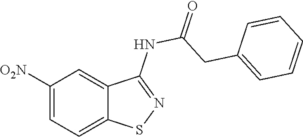 [SMILES: O=C(Cc1ccccc1)Nc1nsc2ccc([N+](=O)[O-])cc12] | >100 | 95.2 ± 1.2 | [symbol] |
| 9 | 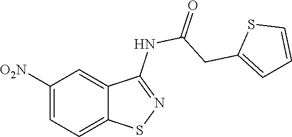 [SMILES: O=C(Cc1cccs1)Nc1nsc2ccc([N+](=O)[O-])cc12] | >100 | 69.0 ± 0.1 | [symbol] |
| 10 | 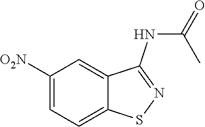 [SMILES: CC(=O)Nc1nsc2ccc([N+](=O)[O-])cc12] | 55.6 ± 3.2 | 90.2 ± 2.7 | [symbol] |
| 11 | 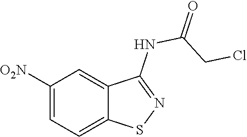 [SMILES: O=C(CCl)Nc1nsc2ccc([N+](=O)[O-])cc12] | 45.3 ± 12.9 | 59.7 ± 2.6 | [symbol] |
| 12 | 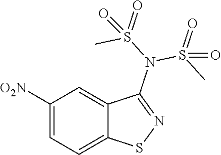 [SMILES: CS(=O)(=O)N(c1nsc2ccc([N+](=O)[O-])cc12)S(C)(=O)=O] | >100 | 63.0 ± 17.4 | [symbol] |
| 13 | 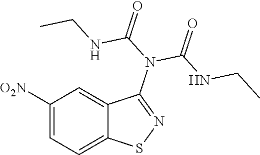 [SMILES: CCNC(=O)N(C(=O)NCC)c1nsc2ccc([N+](=O)[O-])cc12] | 16.4 ± 7.8 | 73.4 ± 1.9 | [symbol] |

Anti-fibrillary effect of additional BTA and 5-nitro-1,2-benzothiazol-3-amine derivatives. To provide more inside about the effect on BTA and 5-nitro-1,2-benzothiazol-3-amine derivatives, thirteen compounds were synthesized and their activity was compared with aniline (i.e., BTA or 5-nitro-1,2-benzothiazol-3-amine) by screening on α-synuclein (α-syn) and TTR₈₁₋₁₂₇ (Table 1). We applied structural modifications on BTA and nitro-1,2-benzothiazol-3-amine cores because they were identified as potent inhibitor of fibrils. The anti-fibrillary and anti-oligomer effects were both evaluated on α-syn. As a starting point, compound 5 is the most related to BTA and previously prepared compounds 1-4. Compounds 5-13 had a weak effect on the aggregation of TTR₈₁₋₁₂₇, with 5-nitro-1,2-benzothiazol-3-amine exhibiting the lowest fluorescence intensity (54.7±1.4%). Compounds 5-9 and 12 did not demonstrate a strong anti-fibrillary activity on α-syn. The 5-nitro-1,2-benzothiazol-3-amine (36.2 t 3.1%), compound 10 (55.6±3.2%), compound 11 (45.3±12.9%), and compound 13 (16.4 t 7.8%) were the best compounds to abrogate α-syn fibril formation. Interestingly, these compounds bear a N-acetamide (compound 10), 2-chloro-N-acetamide (compound 11), or a N-ethyl-1-formamide (compound 13). The presence of larger substituents such as triazine (compounds 6-7), phenyl ring (compound 8), or thiophene (compound 9) led to the significant loss of anti-fibrillary activity. All compounds 5-13 were tested for their anti-oligomer activity on α-syn at a concentration of 50 μM (Table 1). Only the nitro-1,2-benzothiazol-3-amine reduced the oligomer formation using a molar ratio 1:5 (protein:compound).

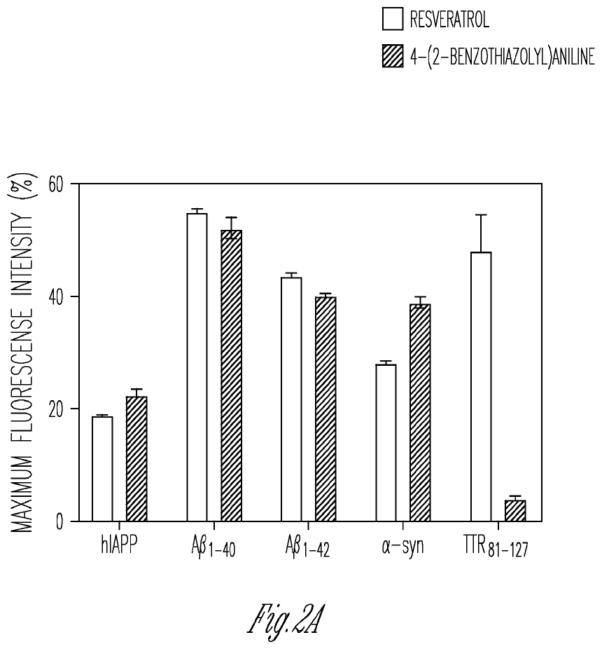

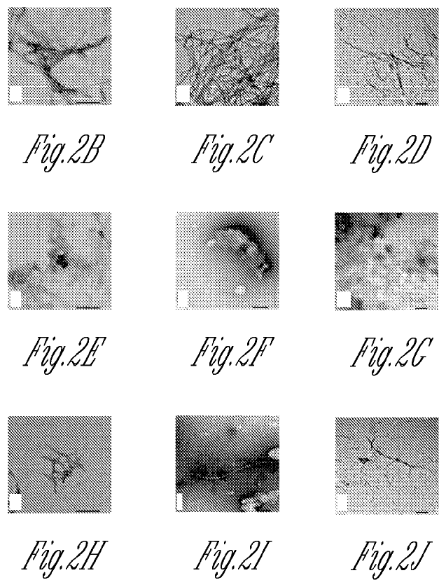

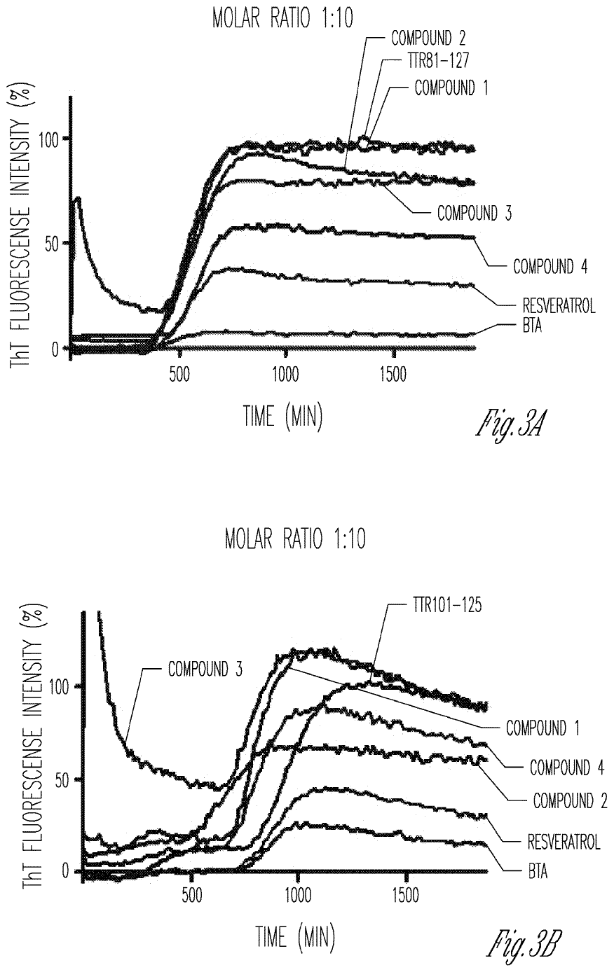

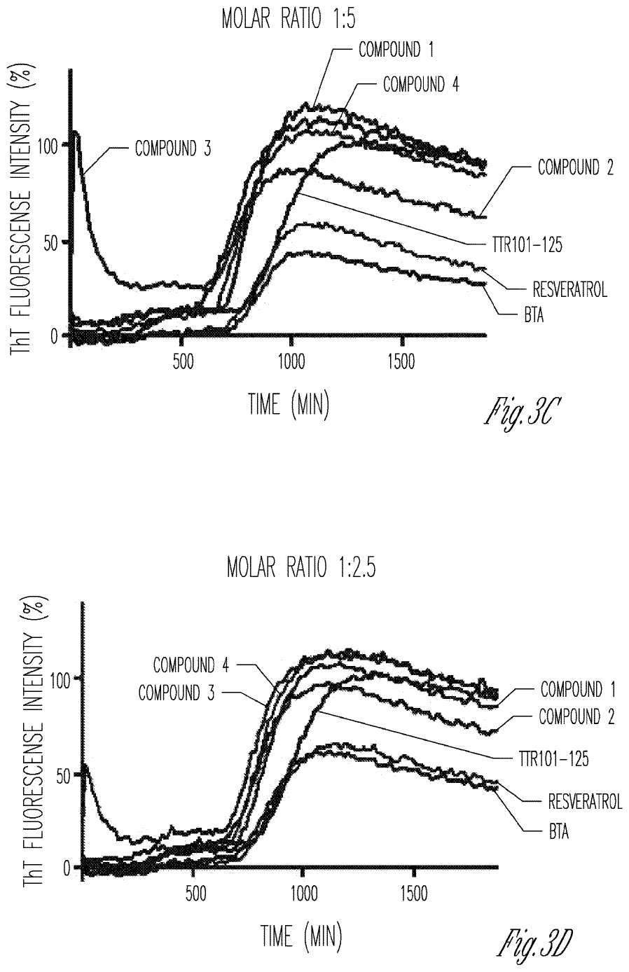

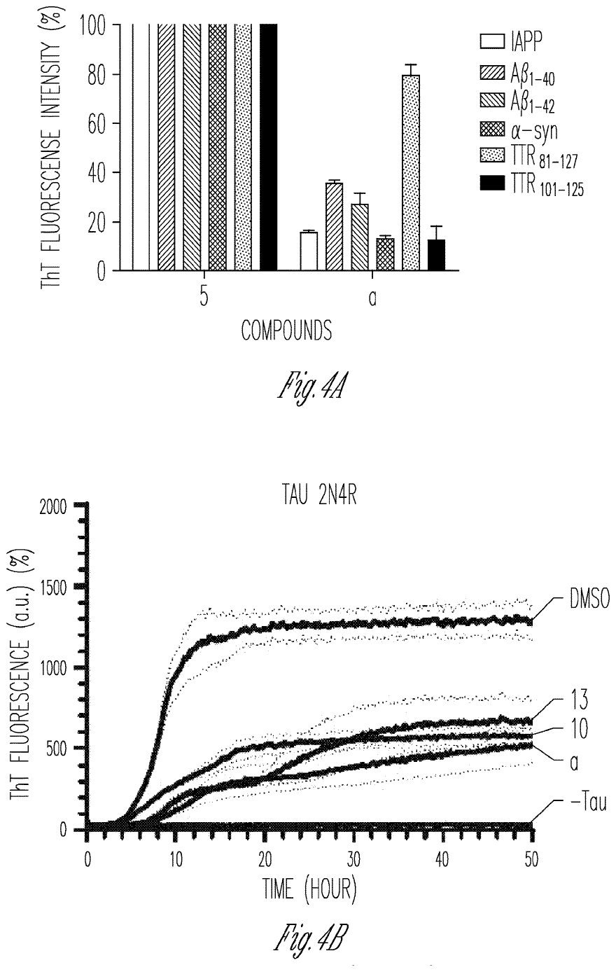

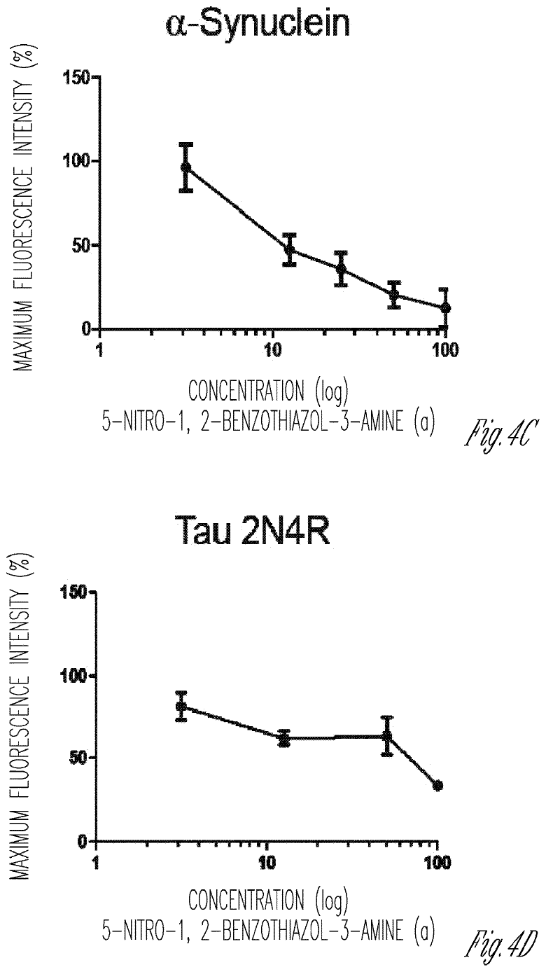

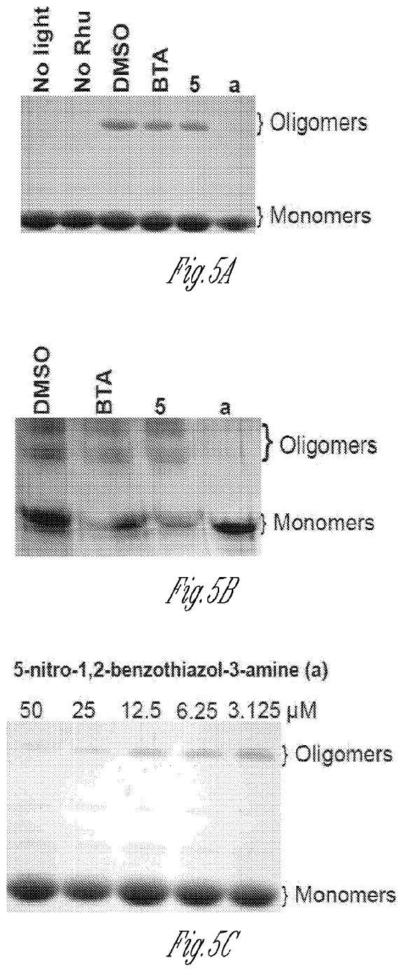

BTA and 5-nitro-1,2-benzothiazol-3-amine exhibited broad anti-fibrillary effect on different prone-to-aggregate proteins. Five important prone-to-aggregate proteins namely, IAPP, Aβ₁₋₄₀, Aβ₁₋₄₂, α-synuclein and TTR (TTR₁₀₁₋₁₂₅ and TTR₈₁₋₁₂₇) were examined for thioflavin T fluorescence intensity (%) using nitro-1,2-benzothiazol-3-amine because of its outstanding anti-oligomer activity (FIG. 4A). Compound 5 produced FI of greater than 100% across all five proteins and was used as negative control. The nitro-1,2-benzothiazol-3-amine was significantly more effective at preventing the aggregation of α-syn, TTR₁₀₁₋₁₂₅, and IAPP. Anti-aggregation activities of nitro-1,2-benzothiazol-3-amine were comparable to BTA (FIG. 2A). Specifically, the Aβ₁₄₂ fluorescence intensity measured of nitro-1,2-benzothiazol-3-amine (27.5±4%) was comparable to BTA (22.59±0.71%). Based on the screen on IAPP, nitro-1,2-benzothiazol-3-amine exhibited an excellent anti-fibrillar activity resulting in a fluorescence intensity of 15.9 t 0.24% and comparable to BTA (11.1±0.3%).

We further examined the anti-aggregation activity of on tau isoform 2N4R using a small selection of compounds because of challenges pertaining to protein procuration. FIG. 4B displays the tau kinetic aggregation curves obtained from a ThT assay of 5-nitro-1,2-benzothiazol-3-amine, compound 10 (intermediate inhibitor of α-syn fibrillization) and compound 13 (best inhibitor of α-syn fibrillization). All reduced the production of tau isoform 2N4R fibrils, 5-nitro-1,2-benzothiazol-3-amine demonstrating the best anti-fibrillary activity. However, only 5-nitro-1,2-benzothiazol-3-amine and compound 13 delayed the lag time. The effect of the lead compound on α-syn and tau aggregation kinetics at lower molar ratios was examined. It was observed that there is a dose-dependent relationship between compound concentration and protein aggregation. Data in FIGS. 4C-4D demonstrated a link between a decrease in fluorescence intensity and the concentration of the 5-nitro-1,2-benzothiazol-3-amine (dose-response). When exposed to increased concentrations of the 5-nitro-1,2-benzothiazol-3-amine, protein fibrillization was reduced in both α-syn and tau isoform 2N4R.

5-nitro-1,2-benzothiazol-3-amine inhibited the formation of α-syn and tau (2N4R) oligomeric species (early-stage of aggregation). To test the effectiveness of the small molecule at the early-stage of aggregation, oligomer formation of α-syn (FIG. 5A) and tau isoform 2N4R (FIG. 5B) were induced in the presence of BTA, 5 (negative control), and 5-nitro-1,2-benzothiazol-3-amine at a concentration of 50 μM by performing a photo-induced cross-linking of unmodified proteins (PICUP) assay. The resulting products after short-light exposure were analyzed with a SDS-PAGE gel stained with Coomassie blue. High molecular weight bands representing the oligomeric species became evident across the two proteins used. 5-nitro-1,2-benzothiazol-3-amine reduced α-syn and tau isoform 2N4R oligomer formation in comparison to the DMSO control (FIG. 5A-B). Results from the PICUP assay indicated that BTA was not successful in preventing oligomerization of the two proteins, despite the inhibition of fibril growth. Compound 5, which was used as a negative control, resulted in high molecular weight bands representing the oligomeric species of α-syn and tau 2N4R. 5-nitro-1,2-benzothiazol-3-amine reduced the oligomer formation in a dose-dependent manner (FIG. 5C-D). The anti-oligomer activity of compound 13 was examined at a higher concentration, since the delay of the lag time was observed in one of the tau ThT assays. Compound 13 reduced substantially the oligomer formation at high concentration, i.e., 200 μM (FIG. 5E). Our candidate 5-nitro-1,2-benzothiazol-3-amine is an effective inhibitor of α-syn and tau oligomerization as shown by its potential to reduce both fibrillization and oligomerization via ThT and PICUP.

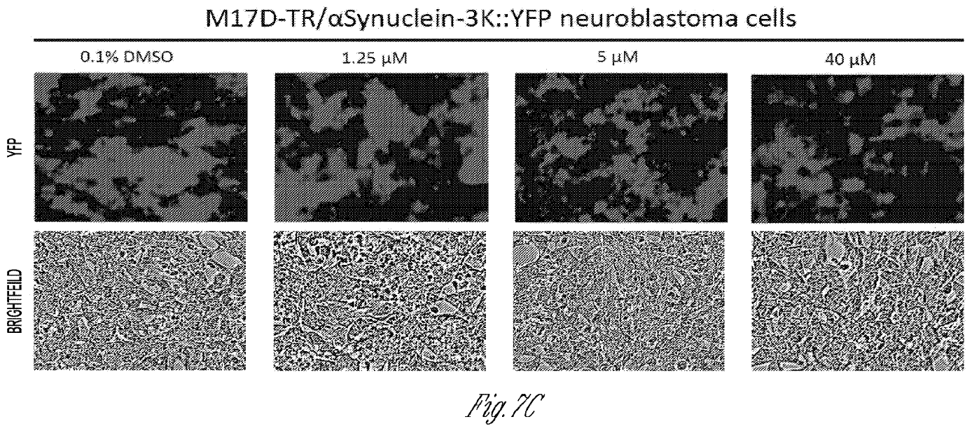

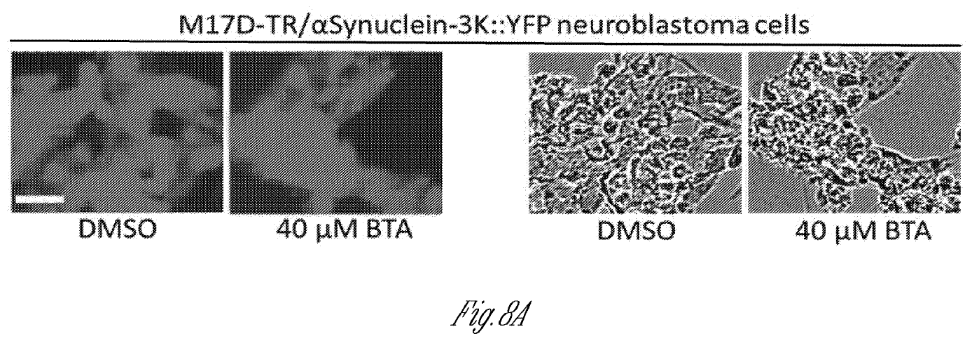

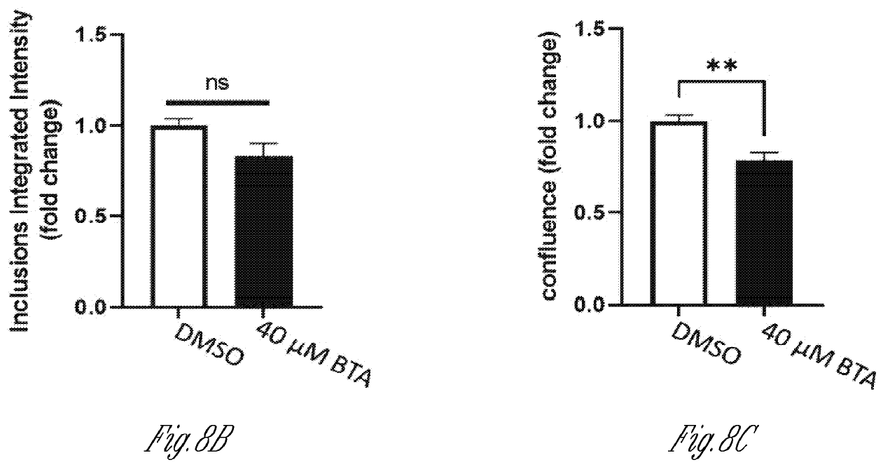

Ultrastructural changes of α-syn and tau isoform 2N4R treated with 5-nitro-1,2-benzothiazol-3-amine. Transmission electron microscopy (TEM) analyses were performed using a solution of α-syn (10 μM) and tau isoform 2N4R (10 μM) after a 24-hour incubation with 0.25% DMSO or 5-nitro-1,2-benzothiazol-3-amine at 100 μM to visualize direct changes in fibril morphology (FIG. 6). 5-nitro-1,2-benzothiazol-3-amine exhibited a clear effect in reducing α-syn and tau fibrillization at a molar ratio of 1:10. In contrast to control α-syn samples treated with DMSO without compound, short fibrils were seen in samples containing 5-nitro-1,2-benzothiazol-3-amine (FIG. 6). The tau fibrils were shorter and less defined when compared to the DMSO control (FIG. 6). TEM data confirm that 5-nitro-1,2-benzothiazol-3-amine was effective at reducing the formation of α-syn and tau isoform 2N4R fibrils.

α-Syn (or αS) inclusion formation and toxicity in neuroblastoma cells. αS E35K+E46K+E61K (=αS3K) ‘amplifies’ the familial-PD-linked αS missense mutation E46K. This model is known to generate round-shaped cytoplasmic inclusions in cultured cells. αS 3K expression leads to cell stress/toxicity which results in a delayed growth of neuroblastoma cells. Using the same system in previous studies, stearoyl-CoA desaturase inhibitors prevented both αS inclusion formation and αS-induced cytotoxicity.

Neuroblastoma cells, M17D, which express an αS-3K::YFP fusion protein in a doxycycline-inducible fashion, were utilized. The α-syn model was used to evaluate the effect of BTA and 5-nitro-1,2-benzothiazol-3-amine on the inclusion formation. 24 h induction of αS-3K: YFP resulted in pronounced round YFP-positive inclusions in the presence of vehicle (DMSO) alone, whereas 5-nitro-1,2-benzothiazol-3-amine reduced the number of inclusions in a dose-dependent manner (starting at 10 μM) without any effect on the cell confluence (FIG. 7). In contrast, BTA (40 μM) did not affect the number of inclusions present in the neuroblastoma cells (FIG. 8). Also, the treatment of BTA led to a reduction in confluence, which is indicative of the compound being slightly toxic at this concentration, in contrast to 5-nitro-1,2-benzothiazol-3-amine.

### CONCLUSION

The inhibitory potential of a series of 13 compounds on the aggregation of IAPP, Aβ₁₋₄₀, Aβ₁₋₄₂, TTR₈₁₋₁₂₇, TTR₀₁₋₁₂₅, α-syn, and tau 2N4R (only with the best compound) was evaluated, with BTA as a control. A ThT fluorescence assay with BTA was performed on five of the prone-to-aggregate proteins (IAPP, Aβ₁₋₄₀, Aβ₁₋₄₂, α-syn, TTR₈₁₋₁₂₇) and validated the formation of fibrils with TEM to demonstrate the effect of BTA in reducing fibrillization. In addition, a ThT assay using transthyretin (TTR₈₁₋₁₂₇, TTR₁₀₁₋₁₂₅) on BTA and the original four compounds at various molar ratios was performed, and it was found that the four compounds were not more effective than BTA in reducing fibrillization. Fibrillization of the remaining compounds was evaluated using ThT, and it was found that only 5-nitro-1,2-benzothiazol-3-amine was effective in reducing α-syn oligomer and fibril formation. Its anti-fibrillar activity is similar to BTA and affected most of the prone-to-aggregate proteins. In contrast to 5-nitro-1,2-benzothiazol-3-amine, BTA didn't inhibit the oligomer formation. A ThT assay and PICUP on tau 2N4R using 5-nitro-1,2-benzothiazol-3-amine were performed, and a reduction in fibrillization and oligomerization were observed. Follow up with a ThT and PICUP dose-response analysis enabled detection of a concentration-dependent effect of 5-nitro-1,2-benzothiazol-3-amine on α-syn and tau 2N4R fibrillization and oligomerization. TEM analysis allowed confirmation of the presence of α-syn and tau 2N4R fibrils when treated with DMSO control, and the reduction of these fibrils when treated with 5-nitro-1,2-benzothiazol-3-amine. Finally, BTA and 5-nitro-1,2-benzothiazol-3-amine were challenged with cell-based assays using M17D neuroblastoma cells expressing inclusion-prone αS-3K::YFP. Only 5-nitro-1,2-benzothiazol-3-amine successfully reduced inclusion in a dose-dependent manner and without affecting cell confluence.

1. A compound comprising a benzisothiazole linked to an amide, a formamide, an amine, or a urea, or a pharmaceutically acceptable salt thereof, wherein at least one carbon on the benzyl ring of the benzisothiazole is substituted with a substituent selected from the group consisting of a C₁-C₆ alkyl, —NO₂, and a halo.

2. The compound of claim 1, or a pharmaceutically acceptable salt thereof, wherein the C₁-C₆ alkyl is methyl.

3. The compound of claim 1, or a pharmaceutically acceptable salt thereof, wherein the halo is F, Cl, or Br.

4. The compound of claim 1, wherein the compound is a compound of the formula: 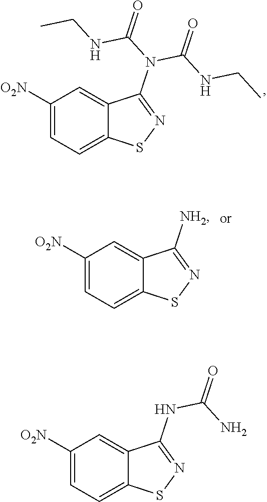 [SMILES: CCNC(=O)N(C(=O)NCC)c1nsc2ccc([N+](=O)[O-])cc12 ; Nc1nsc2ccc([N+](=O)[O-])cc12 ; NC(=O)Nc1nsc2ccc([N+](=O)[O-])cc12]
or a pharmaceutically acceptable salt thereof.

5. (canceled)

6. (canceled)

7. The compound of claim 1, wherein the compound is a compound of the formula: 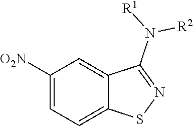
or a pharmaceutically acceptable salt thereof,
wherein:
each R₁ and R₂ is independently selected from hydrogen, alkyl, cycloalkyl, aryl, heteroaryl, —SO₂R, and —COR, wherein R₁ and R₂ are each, independently, optionally substituted with an alkyl, a nitro, a cyano, a halo, an amino, a hydroxy, an alkoxy, an aryl, or a heteroaryl, and wherein R is an alkyl, an alkenyl, an alkynyl, a cycloalkyl, a heterocycloalkyl, an aryl, or a heteroaryl, and wherein R is optionally substituted with a halo, an amino, a nitro, a cyano, a hydroxy, an alkoxy, an aryl, or a heteroaryl group.

8. The compound of claim 7, wherein the compound is a compound of the formula: 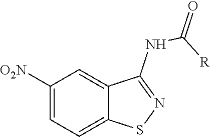
or a pharmaceutically acceptable salt thereof,
wherein:
R is an alkyl, an alkenyl, an alkynyl, a cycloalkyl, a heterocycloalkyl, an aryl, or a heteroaryl, and wherein R is optionally substituted with a halo, an amino, a nitro, a cyano, a hydroxy, an alkoxy, an aryl, or a heteroaryl group.

9. (canceled)

10. (canceled)

11. A compound of the formula: 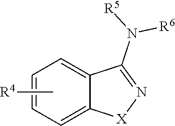
or a pharmaceutically acceptable salt thereof;
wherein:
X is O, NR³, or S, wherein R³ is H or alkyl;
R⁴ is an electron withdrawing group, and
each R⁵ and R⁶ is independently selected from hydrogen, alkyl, cycloalkyl, aryl, heteroaryl, —SO₂R, —C(O)NHR, and —C(NR⁷)NHR, wherein R⁵ and R⁶ can each be independently optionally substituted with an alkyl, a nitro, a cyano, a halo, an amino, a hydroxy, an alkoxy, an aryl, or a heteroaryl, and R is an alkyl, an alkenyl, an alkynyl, a cycloalkyl, a heterocycloalkyl, an aryl, or a heteroaryl, and wherein R can be optionally substituted with a halo, an amino, a nitro, a cyano, a hydroxy, an alkoxy, an aryl, or a heteroaryl group.

12. The compound of claim 11, wherein the compound is a compound of the formula: 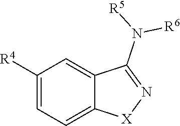
or a pharmaceutically acceptable salt thereof.

13. A compound of the formula: 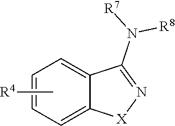
or a pharmaceutically acceptable salt thereof;
wherein:
X is O, NR³, or S, wherein R³ is H or alkyl;
R⁴ is an electron withdrawing group; and
each R⁷ and R⁸ is independently selected from H, —C(O)NHR, alkylaminoalkyl, aminoalkyl, arylamino, heteroarylamino, alkyl substituted with at least one group comprising a hydrogen atom capable of hydrogen bonding with at least one hydrogen bonding acceptor, aryl substituted with at least one group comprising a hydrogen atom capable of hydrogen bonding with a hydrogen bonding acceptor, heteroaryl substituted with at least one group comprising a hydrogen atom capable of hydrogen bonding with a hydrogen bonding acceptor, and heteroaryl comprising at least one hydrogen atom capable of hydrogen bonding with a hydrogen bonding acceptor, each of which can independently optionally be substituted with an alkyl, a nitro, a cyano, a halo, an amino, a hydroxy, an alkoxy, an aryl, or a heteroaryl, and R is an alkyl, an alkenyl, an alkynyl, a cycloalkyl, a heterocycloalkyl, an aryl, or a heteroaryl and wherein R can be optionally substituted with a halo, an amino, a nitro, a cyano, a hydroxy, an alkoxy, an aryl, or a heteroaryl group.

14. (canceled)

15. The compound of claim 13, or a pharmaceutically acceptable salt thereof, wherein R⁷ is H and R⁸ is selected from H, —C(O)NHR, alkylaminoalkyl, aminoalkyl, arylamino, and heteroarylamino, each of which can be optionally substituted with a halo, an amino, a nitro, a cyano, a hydroxy, an alkoxy, an aryl, or a heteroaryl group.

16. The compound of claim 13, or a pharmaceutically acceptable salt thereof, wherein the group comprising a hydrogen atom capable of hydrogen bonding with a hydrogen bonding acceptor is —C(O)OH, —C(O)NHR, or —OH.

17. The compound of claim 13, or a pharmaceutically acceptable salt thereof, wherein the heteroaryl comprising at least one hydrogen atom capable of hydrogen bonding with a hydrogen bonding acceptor is selected from:

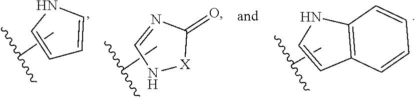

18. The compound of claim 11, or a pharmaceutically acceptable salt thereof, wherein the electron withdrawing group is selected from —NC(O)R″, —NSO₂R″, —C(O)R″, —CN, —NO₂, —CX¹₃, X¹, —C(O)OR″, —C(O)NH₂, —C(O)NR″₂, —C(O)R″, —C(O)X, —S(O)₂R″, —S(O)₂OR″, —SO₃H₂, —S(O)₂NH₂, —S(O)₂NR″₂, —PO₃H₂, —P(O)(OR″)₂, —NO, —NH₂, —NR₂″, —N(R″)₃⁺, and salts thereof, wherein X¹ is —F, —Br, —Cl, or —I, and each R″ is, at each occurrence, independently selected from H, C₁₋₆ alkyl and aryl.

19. The compound of claim 11, or a pharmaceutically acceptable salt thereof, wherein the electron withdrawing group is selected from halo, —NO₂, —CN, —CF₃, or —C(O)R″, wherein R″ can be H, C₁₋₆ alkyl and aryl.

20. A pharmaceutical composition comprising at least one compound of claim 1, or a pharmaceutically acceptable salt thereof, and a pharmaceutically acceptable carrier.

21. A method of inhibiting protein aggregation in a subject having, or at risk for, aggregation of a protein prone to aggregate in a state of disease, which method comprises administering to the subject the composition of claim 20 in an amount effective to inhibit protein aggregation, whereupon protein aggregation is inhibited in the subject having, or at risk for, aggregation of a protein prone to aggregate in a state of disease.

22. The method of claim 21, wherein the protein prone to aggregate is islet amyloid polypeptide, amyloid-β, α-synuclein, tubulin associated unit (tau), or transthyretin.

23. The method of claim 22, wherein the tau is tau isoform 2N4R or 1N4R or 0N4R, 2N3R or 1N3R or 0N3R with or without post-translational changes.

24. The method of claim 21, wherein the disease is AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis.

25. The method of claim 21, wherein the subject has, or is at risk for, Alzheimer's disease or Parkinson's disease.

26. (canceled)
